(19) United States
(12) Patent Application Publication
(10) Pub. No.: US 2012/0010496 A1
(43) Pub. Date: Jan. 12, 2012

(54) MEDICAL IMAGE DIAGNOSIS APPARATUS AND A CONTROLLING METHOD

(76) Inventor: Naoyuki FURUDATE, Otawara-shi (JP)

(21) Appl. No.: 13/177,782

(22) Filed: Jul. 7, 2011

(30) Foreign Application Priority Data
Jul. 9, 2010 (JP) 2010-156631
Jun. 13, 2011 (JP) 2011-131290

(51) Int. Cl. A61B 5/055

(52) U.S. Cl. 600/410

(57) ABSTRACT

A medical image diagnosis apparatus according to the present embodiments includes a storage and an execution controller. The storage is configured to store therein a program for executing a plurality of processes contained in an image taking procedure or a plurality of processes contained in a post-processing procedure, while the processes are classified into a first group for which an input operation from an operator is received and a second group for which the input operation is not received, and the processes are associated with one another according to an order. The execution controller is configured to exercise control so that the processes are executed according to the order. When executing a process classified into the first group, the program displays information selected according to a purpose of the image taking procedure or the post-processing procedure, as an operation screen.

### CROSS-REFERENCE TO RELATED APPLICATIONS

This application is based upon and claims the benefit of priority from Japanese Patent Application No. 2010-156631, filed on Jul. 9, 2010; and Japanese Patent Application No. 2011-131290, filed on Jun. 13, 2011, the entire contents of all of which are incorporated herein by reference.

### FIELD

Embodiments described herein relate generally to a medical image diagnosis apparatus and a controlling method.

### BACKGROUND

Generally speaking, an image taking procedure using a Magnetic Resonance Imaging (MRI) apparatus involves complicated operations. For this reason, MRI apparatuses provide a Graphical User Interface (GUI) with regard to operations for which an input of information is received from the operator, so that the input can be received through the GUI.

For example, an MRI apparatus displays parameters corresponding to various functions of the MRI apparatus, on an image taking condition editing screen. Accordingly, the operator selects one ore more parameters being setting targets out of the parameters displayed on the image taking condition editing screen and configure settings.

However, operators who use MRI apparatuses on a daily basis often feel that the operations that are actually performed in order for the operators to execute the image taking action are cumbersome. For example, when performing an input operation through a GUI, the operator finds it bothersome that there are many other parameters beside the parameter of which the setting is actually configured. This problem is not limited to the image taking procedure employing an MRI apparatus, but also similarly applies to image taking procedures employing other medical image diagnosis apparatuses. For this reason, there is a demand for improvements of operability during image taking procedures employing medical image diagnosis apparatuses.

### BRIEF DESCRIPTION OF THE DRAWINGS

FIG. 1 is a block diagram of an MRI apparatus according to a first embodiment;

FIG. 2 is a drawing for explaining execution control of a program according to the first embodiment;

FIG. 3 is a block diagram of a computer system according to the first embodiment;

FIGS. 4 to 30 are drawings for explaining a scenario and an execution of the scenario according to the first embodiment;

FIG. 31 is a block diagram of a controller according to a second embodiment;

FIG. 32 is a drawing for explaining a scenario according to another embodiment; and

FIGS. 33 to 35 are drawings for explaining exemplary configurations according to other embodiments.

### DETAILED DESCRIPTION

The MRI apparatus according to the present embodiments includes a storage and an execution controller. The storage is configured to store therein a program for executing a plurality of processes contained in an image taking procedure or a plurality of processes contained in a post-processing procedure performed on data acquired during the image taking procedure, while the processes are classified into a first group for which an input operation from an operator is received and a second group for which the input operation is not received, and the processes are associated with one another according to an order in which the processes are to be executed during the image taking procedure or the post-processing procedure. The execution controller is configured to exercise control so that, when a start instruction to start the image taking procedure or the post-processing procedure is received, the execution of the program is started and the processes are executed according to the order. When executing a process classified into the first group, the program displays, on a display unit, information selected according to a purpose of the image taking procedure or the post-processing procedure, as an operation screen for receiving the input operation.

In the following sections, as exemplary embodiments of a medical image diagnosis apparatus, MRI apparatuses according to a first embodiment and a second embodiment will be explained. It is possible to apply the technical features disclosed herein not only to an image taking procedure employing an MRI apparatus, but also to an image taking procedure employing other medical image diagnosis apparatuses such as an X-ray diagnosis apparatus or an X-ray Computed Tomography (CT) apparatus.

First, an MRI apparatus 100 according to the first embodiment will be briefly explained. The MRI apparatus 100 according to the first embodiment includes: an image taking unit; a storage; a receiving unit; and a controller.

The image taking unit is, for example, a sequence controlling unit 10 explained below. The image taking unit sequentially performs a plurality of types of image taking procedures on an examined subject (hereinafter, the “patient”). The storage is, for example, a scenario storage 23b explained below. The storage stores therein a plurality of medical examination flows as clinical application scenarios (CASs) (hereinafter, simply referred to as “scenarios” when appropriate) in which the plurality of types of image taking procedures are arranged in an order. The storage also stores therein a plurality of image taking conditions necessary for executing the image taking procedures, while classifying the image taking conditions into image taking conditions for which an input operation from an operator of the apparatus (hereinafter, “operator”) is received and image taking conditions for which an input operation is not received. Further, the storage stores therein timing with which an operation screen for receiving the input operation is displayed during the medical examination flows. The receiving unit is, for example, an image-taking start instruction receiving unit 26a explained below. The receiving unit receives, from the operator, a start instruction to start a specified one of the plurality of clinical application scenarios. The controller is, for example, a scenario controller 26b explained below. When having received the start instruction, the controller displays an operation screen for receiving an input operation at the stored timing, while any of the medical examination flows is being executed. The controller also ensures that an image taking parameter of the image taking conditions set by the input operation and the image taking conditions for which an input operation is not received are reflected in an image taking procedure performed after an input is made on the operation screen.

Further, in the first embodiment, the clinical application scenario is a medical examination flow for sequentially executing: a pilot scan for determining a position; a prep scan for determining a delay period from an R wave; and a non-contrast-enhanced Magnetic Resonance Angiography (MRA) scan for performing an image taking procedure when the determined delay period has elapsed. Further, in the first embodiment, the scenario controller 26b displays, on an operation screen, information for determining an image taking position including a position determining image obtained by the pilot scan, at a time after the pilot scan and before the prep scan. Further, the scenario controller 26b displays, on an operation screen, information for supporting the determination of the delay period from the R wave, at a time after the prep scan and before the non-contrast-enhanced MRA scan.

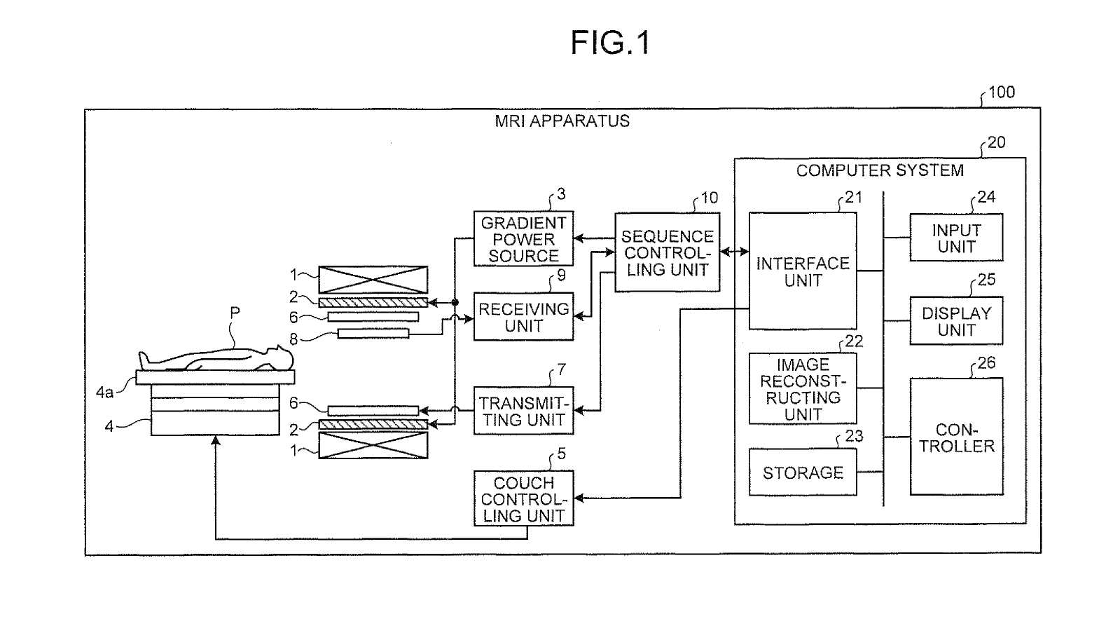

Next, a configuration of the MRI apparatus 100 according to the first embodiment will be explained, with reference to FIG. 1. FIG. 1 is a block diagram of the MRI apparatus 100 according to the first embodiment. As shown in FIG. 1, the MRI apparatus 100 according to the first embodiment includes: a magnetostatic field magnet 1, a gradient coil 2, a gradient power source 3, a couch 4, a couch controlling unit 5, a transmission coil 6, a transmitting unit 7, reception coil 8, a receiving unit 9, the sequence controlling unit 10, and a computer system 20.

The magnetostatic field magnet 1 is formed in the shape of a hollow circular cylinder and generates a uniform magnetostatic field in the space on the inside thereof. The magnetostatic field magnet 1 may be configured by using, for example, a permanent magnet, a superconductive magnet, or the like. The gradient coil 2 is formed in the shape of a hollow circular cylinder and generates a gradient magnetic field in the space on the inside thereof. More specifically, the gradient coil 2 is disposed on the inside of the magnetostatic field magnet 1 and generates the gradient magnetic field by receiving a supply of electric current from the gradient power source 3. The gradient power source 3 supplies the electric current to the gradient coil 2 according to pulse sequence execution data transmitted thereto from the sequence controlling unit 10.

The couch 4 includes a couchtop 4a on which a patient P is placed. While the patient P is placed thereon, the couchtop 4a is inserted into the hollow (i.e., an image taking aperture) of the gradient coil 2. Normally, the couch 4 is provided so that the longitudinal direction thereof extends parallel to the central axis of the magnetostatic field magnet 1. The couch controlling unit 5 drives the couch 4 so that the couchtop 4a moves in the longitudinal direction and in an up-and-down direction.

The transmission coil 6 generates a radio-frequency magnetic field. More specifically, the transmission coil 6 is disposed on the inside of the gradient coil 2 and generates the radio-frequency magnetic field by receiving a supply of a radio-frequency pulse from the transmitting unit 7. The transmitting unit 7 transmits the radio-frequency pulse corresponding to a Larmor frequency to the transmission coil 6, according to pulse sequence execution data transmitted thereto from the sequence controlling unit 10.

The reception coil 8 receives an echo signal. More specifically, the reception coil 8 is disposed on the inside of the gradient coil 2 and receives the echo signal emitted from the patient P due to an influence of the radio-frequency magnetic field. Further, the reception coil 8 outputs the received echo signal to the receiving unit 9. For example, the reception coil 8 may be a reception coil for the head of the patient, a reception coil for the spine, or a reception coil for the abdomen.

Based on the echo signal being output from the reception coil 8, the receiving unit 9 generates echo signal data according to pulse sequence execution data transmitted thereto from the sequence controlling unit 10. More specifically, the receiving unit 9 generates the echo signal data by applying a digital conversion to the echo signal being output from the reception coil 8 and transmits the generated echo signal data to the computer system 20 via the sequence controlling unit 10. The receiving unit 9 may be provided on the side where a gantry device is provided, the gantry device including the magnetostatic field magnet 1 and the gradient coil 2.

The sequence controlling unit 10 controls the gradient power source 3, the transmitting unit 7, and the receiving unit 9. More specifically, the sequence controlling unit 10 transmits the pulse sequence execution data transmitted thereto from the computer system 20, to the gradient power source 3, to the transmitting unit 7, and to the receiving unit 9.

The computer system 20 includes an interface unit 21, an image reconstructing unit 22, a storage 23, an input unit 24, a display unit 25, and a controller 26. The interface unit 21 is connected to the sequence controlling unit 10 and controls inputs and outputs of data that is transmitted and received between the sequence controlling unit 10 and the computer system 20. The image reconstructing unit 22 reconstructs image data from the echo signal data transmitted thereto from the sequence controlling unit 10 and stores the reconstructed image data into the storage 23.

The storage 23 stores therein the image data stored by the image reconstructing unit 22 and other data used in the MRI apparatus 100. For example, the storage 23 is configured by using a semiconductor memory element such as a Random Access Memory (RAM) or a flash memory, a hard disk, an optical disk, or the like.

The input unit 24 receives an image-taking start instruction and editing to image taking conditions from the operator. For example, the input unit 24 may be configured by using any of the following: a pointing device such as a mouse and/or a trackball; a selecting device such as a mode changing switch; and an input device such as a keyboard. The display unit 25 displays the image data, an image taking condition editing screen, and the like. For example, the display unit 25 may be a display device such as a liquid crystal display monitor.

The controller 26 exercises overall control of the MRI apparatus 100 by controlling the functional units described above. For example, the controller 26 may be an integrated circuit such as an Application Specific Integrated Circuit (ASIC) or a Field Programmable Gate Array (FPGA) or an electronic circuit such as a Central Processing Unit (CPU) or a Micro Processing Unit (MPU).

The MRI apparatus 100 according to the first embodiment stores therein a computer program (hereinafter, “program”) for executing a plurality of processes contained in an image taking procedure. Further, when having received an image-taking start instruction, the MRI apparatus 100 exercises control so that the processes included in the program are executed according to the order in which the processes are to be executed during the image taking procedure. This function is mainly realized by the computer system 20 in the first embodiment.

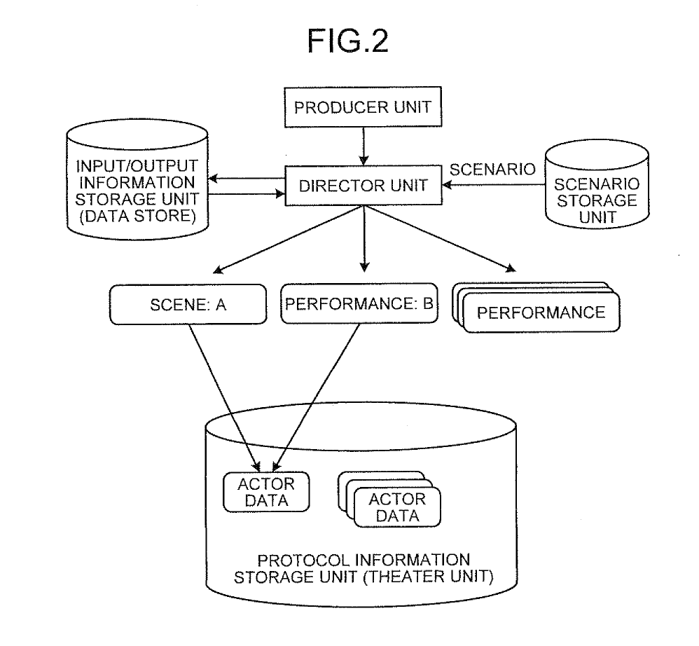

FIG. 2 is a drawing for explaining execution control of the program according to the first embodiment. The MRI apparatus 100 according to the first embodiment defines the program for executing the plurality of processes contained in the image taking procedure as a “scenario”. In the “scenario”, the processes contained in the image taking procedure are classified into “scenes” for which an input operation from the operator is received and “performances” for which an input operation is not received. The processes are associated with one another according to the order in which the processes are to be executed during the image taking procedure. The “scenes” are, so to speak, processes that involve interactions with the operator.

Further, the MRI apparatus 100 according to the first embodiment defines a collection of pieces of information related to protocols as a “theater unit (hereinafter, “theater”)”. Also, the MRI apparatus 100 defines the pieces of information related to the protocols included in the “theater” as “actor data (hereinafter, “actor”)”. Examples of the information related to the protocols include: information that is set in advance for controlling a protocol included in an image taking procedure, information that is set after receiving an input operation from the operator, and image data acquired during an image taking procedure. In other words, the information related to the protocols include information that is set in advance and information that is set or acquired in an post-event manner, with respect to any of the protocols.

The MRI apparatus 100 according to the first embodiment defines functions that control the execution of a “scenario” as a “producer unit (hereinafter, “producer”)” and a “director unit (hereinafter, “director”)”. More specifically, the “producer” exercises an overall control, whereas the “director” controls the “scenario”. In other words, according to the first embodiment, each of the “scenarios” is defined according to the purpose of an image taking procedure, whereas a “director” is defined for each of the “scenarios” that are defined according to the purposes of the image taking procedures, respectively. Accordingly, the “producer” controls the plurality of “scenarios” by controlling pairs each of which is made up of a “director” and a “scenario”.

Next, a relationship among the “scenario”, the “scenes”, the “performances”, the “theater”, the “actor”, the “producer”, and the “director” will be explained with reference to FIG. 2. As shown in FIG. 2, the MRI apparatus 100 according to the first embodiment has stored, in advance, a “scenario” defined according to the purpose of an image taking procedure in the scenario storage, which is explained later. The “scenario” is stored in advance in the scenario storage in the form of a file written in, for example, the Extensible Markup Language (XML). The operator is able to edit the “scenario” stored in the scenario storage and is able to, for example, modify the contents of any of the processes according to the purpose of the image taking procedure or modify the execution order.

Further, the MRI apparatus 100 has the “actor” stored in the protocol information storage, which is explained later. As shown in FIG. 2, the “actor” are input/output data of the “scenes” and the “performances”. In other words, the protocol information storage stores therein, in advance, the “actor” to be used in the “scenes” and the “performances” and also stores therein the “actor” generated by the “scenes” and the “performances”. The protocol information storage corresponds to the “theater”.

Further, as shown in FIG. 2, the “producer”, which is one of the functions of the scenario controller (explained later) starts up the “director”, which is another function of the scenario controller, so that the “director” reads the “scenario” from the scenario storage. The “scenario” includes the “scenes” and the “performances” as the plurality of processes contained in the image taking procedure. Accordingly, as shown in FIG. 2, the “director” sequentially starts up the processes of the “scenes” and the “performances”, so that the processes of the “scenes” and the “performances” are executed according to the order in which these processes are to be executed during the image taking procedure.

Further, as shown in FIG. 2, the MRI apparatus 100 according to the first embodiment includes an input/output information storage, which is explained later. The MRI apparatus 100 defines the input/output information storage as a “data store”. Further, the MRI apparatus 100 performs a data exchange between the “producer” and the “director”, a data exchange between the “director” and a “scene”, and a data exchange between the “director” and a “performance”, via the “data store”.

The data structure of the data exchanged via the “data store” may be a set made up of a “keyword” and “data” or a set made up of a “keyword” and “data”, where the “data” itself is a plurality of sets each made up of a “keyword” and “data”. The “scenario” is written, in advance, in such a manner that the data exchange between the “producer” and the “director”, the data exchange between the “director” and the “scenes”, and the data exchange between the “director” and the “performances” are performed in the corresponding data structure.

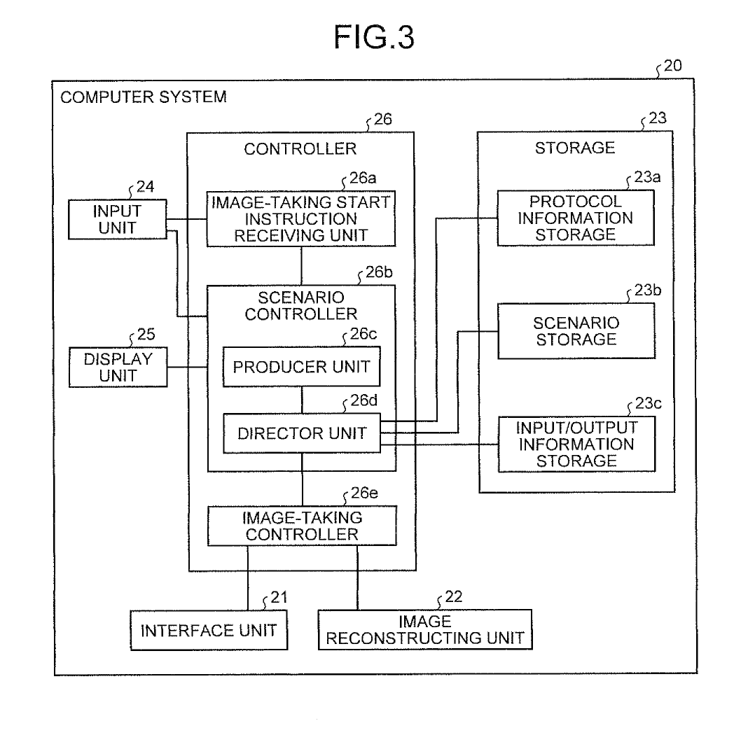

FIG. 3 is a block diagram of the computer system 20 according to the first embodiment. As shown in FIG. 3, the storage 23 according to the first embodiment includes a protocol information storage 23a, the scenario storage 23b, and an input/output information storage 23c. The protocol information storage 23a corresponds to the “theater” described above. The input/output information storage 23c corresponds to the “data store” described above.

Further, as shown in FIG. 3, the controller 26 according to the first embodiment includes the image-taking start instruction receiving unit 26a, the scenario controller 26b, and an image-taking controller 26e. The image-taking start instruction receiving unit 26a receives an image-taking start instruction to start an image taking procedure, from the operator via the input unit 24.

When the image-taking start instruction receiving unit 26a receives an image-taking start instruction, the scenario controller 26b starts the execution of the “scenario” and exercises control so that the processes contained in the “scenario” are executed according to the order in which the processes are to be executed during the image taking procedure. More specifically, the scenario controller 26b includes a producer 26c and a director 26d. The producer 26c and the director 26d are each an integrated circuit such as an ASIC or an FPGA or an electronic circuit such as a CPU or an MPU. The producer 26c corresponds to the “producer” described above. The director 26d corresponds to the “director” described above. When executing a “scene”, for example, the director 26d displays an operation screen for receiving an input operation from the operator, on the display unit 25. On the operation screen, information selected according to the purpose of the image taking procedure is displayed, as information that is necessary for receiving the input operation. It means that a GUI exclusively for the “scene” is displayed. Further, the director 26d receives the input operation from the operator via the input unit 24 and, if a “Next” button is pressed instead of a “Save” button, for example, the director 26d executes the process at the following stage according to the order.

The image-taking controller 26e controls the image taking procedure. For instance, when the director 26d executes a “scene” or a “performance” so that a process to control, for example, the gradient power source 3, the transmitting unit 7, and the receiving unit 9 is executed, the image-taking controller 26e controls the gradient power source 3, the transmitting unit 7, and the receiving unit 9, via the interface unit 21. As another example, when the director 26d executes a “scene” or a “performance” so that an image reconstructing process by the image reconstructing unit 22 is performed, the image-taking controller 26e controls the image reconstructing unit 22.

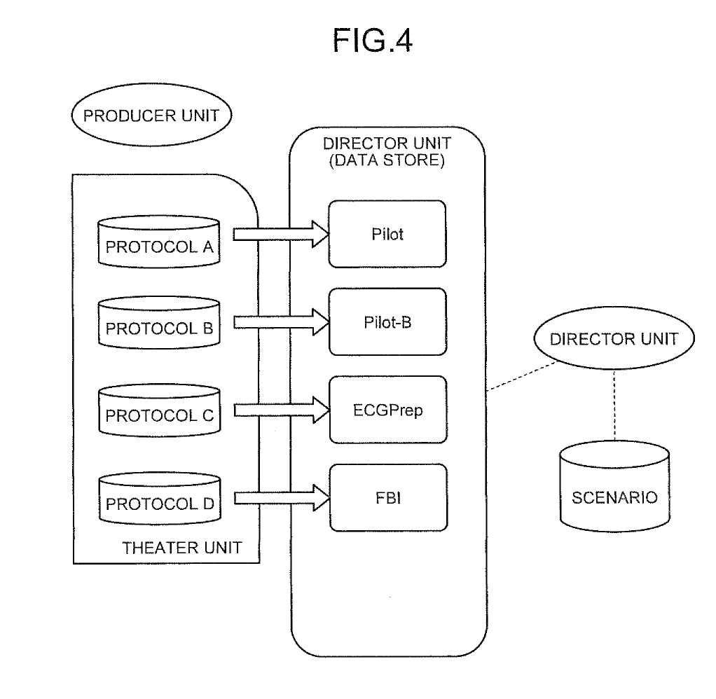

Next, a processing procedure performed by the MRI apparatus 100 according to the first embodiment will be explained with reference to FIGS. 4 to 30. In the following sections, an example in which the purpose of the image taking procedure is to perform an “image taking procedure on a leg by using a Fresh Blood Imaging (FBI) method” will be explained; however, the disclosed technical features are not limited to this example. The purpose of the image taking procedure can be arbitrarily modified with any other image taking method and/or any other site used as the image taking target.

Next, the FBI method will be briefly explained. The FBI method is an example of a non-contrast-enhanced Magnetic Resonance (MR) blood vessel image taking method by which a three-dimensional image is obtained while using an electrocardiographic synchronization or a pulse-wave synchronization. More specifically, according to the FBI method, blood vessels are rendered without administering a contrast agent and by scanning a bloodstream that is pumped out from the heart in correspondence with each cardiac phase and that is fresh, stable, and has a high flow rate. For example, in synchronization with signals that express the cardiac phases of the patient and are acquired by an electrocardiograph or an electroencephalograph, the MRI apparatus 100 repeats an operation to acquire an echo signal group corresponding to a predetermined number of three-dimensional slice encodes (e.g., one slice encode), by performing the operation once every two or more heart beats (e.g., 2-5 R-R). In other words, a long repetition time (TR) is used. The Echo Time (TE) is also a long TE. The TE and the TR are each set to be in a range where it is possible to obtain a T2-highlighted image in which the T2 component of the blood is highlighted. The MRI apparatus 100 repeats the operation to acquire the echo signal group by performing the operation once every two heart beats for a patient having a low heart rate (HR) or once every five heart beats for a patient having a high HR.

In an image taking procedure employing the FBI method, to obtain an image having an excellent rendering resolution of the blood vessels while applying an electrocardiographic synchronization thereto, it is desirable to set an image taking condition so that the echo signal emitted from the patient becomes the strongest. It is known that the strength of the echo signal depends on the delay period from the R wave. For this reason, by performing a preparatory image taking procedure, the MRI apparatus 100 determines an optimal delay period so that the image taking procedure is performed when the predetermined delay period has elapsed after the R wave. For example, an electrocardiogram(ECG)-Prep image taking procedure is a preparatory image taking procedure that is performed while varying the delay period so as to determine the optimal delay period and so as to obtain a two-dimensional image by an electrocardiographic synchronization or a pulse-wave synchronization. During the ECG-Prep image taking procedure, images are taken a plurality of times, while using mutually-different delay periods. For example, the MRI apparatus 100 performs a delay period determining process in the following manner: The MRI apparatus 100 displays a plurality of two-dimensional images on the display unit 25 and prompts the operator to select one of the two-dimensional images. The MRI apparatus 100 then determines the delay period used for obtaining the two-dimensional image selected by the operator, as the delay period to be used in the image taking procedure employing the FBI method. As another example, the MRI apparatus 100 applies image processing to a plurality of two-dimensional images so as to determine the delay period obtained as a result of the image processing as the delay period to be used in the image taking procedure employing the FBI method.

For this reason, the first embodiment will be explained on the assumption that the “image taking procedure performed on a leg by using the FBI method” includes a pilot image taking procedure, a plan image taking procedure, the ECG-Prep image taking procedure, and an FBI image taking procedure. FIGS. 4 to 30 are drawings for explaining a scenario and an execution of the scenario according to the first embodiment.

First, to start the “image taking procedure performed on a leg by using the FBI method”, the operator of the MRI apparatus 100 sets an area near an ankle of the patient as the center of a magnetic field. When performing the image taking procedure on a leg by using the FBI method, the couch 4 sequentially moves from the abdomen toward the feet of the patient, so that the area near the ankle is a final moving position.

After that, the operator of the MRI apparatus 100 operates the computer system 20, specifies a “scenario” via the input unit 24, and instructs that the image taking procedure should be started. In the first embodiment, the “scenario” is arranged so that the purpose of the image taking procedure is to perform the “image taking procedure on a leg by using the FBI method”. Accordingly, the image-taking start instruction receiving unit 26a receives the image-taking start instruction from the operator and instructs the “producer” in the scenario controller 26b to start the “image taking procedure performed on a leg by using the FBI method”.

The explanation will continue with reference to FIG. 4. The “producer” specifies the “scenario” and starts up the “director”. Further, according to the program written in the “scenario”, the “producer” reads a protocol (i.e., a protocol A) for the pilot image taking procedure that is stored in the “theater” in advance and stores a title ‘Pilot’ used for treating the read protocol A as an “actor” into the “data store”. In other words, the data itself of the protocol A is stored in the “theater”. The title ‘Pilot’ registered in the “data store” is used for keeping the “actor” having the keyword ‘Pilot’ associated with the data of the protocol A stored in the “theater”.

Further, according to the program written in the “scenario”, the “producer” reads a protocol (i.e., a protocol B) for the plan image taking procedure that is stored in the “theater” in advance and stores a title ‘Pilot-2’ used for treating the read protocol B as an “actor” into the “data store”. In other words, the data itself of the protocol B is stored in the “theater”. The title ‘Pilot-B’ registered in the “data store” is used for keeping the “actor” having the keyword ‘Pilot-B’ associated with the data of the protocol B stored in the “theater”.

Further, according to the program written in the “scenario”, the “producer” reads a protocol (i.e., a protocol C) for the ECG-Prep image taking procedure that is stored in the “theater” in advance and stores a title ‘ECGPrep’ used for treating the read protocol C as an “actor” into the “data store”. In other words, the data itself of the protocol C is stored in the “theater”. The title ‘ECGPrep’ registered in the “data store” is used for keeping the “actor” having the keyword ‘ECGPrep’ associated with the data of the protocol C stored in the “theater”.

Further, according to the program written in the “scenario”, the “producer” reads a protocol (i.e., a protocol D) for the FBI image taking procedure that is stored in the “theater” in advance and stores a title ‘FBI’ used for treating the read protocol D as an “actor” into the “data store”. In other words, the data itself of the protocol D is stored in the “theater”. The title ‘FBI’ registered in the “data store” is used for keeping the “actor” having the keyword ‘FBI’ associated with the data of the protocol D stored in the “theater”.

In the manner described above, the “data store” stores therein a data set having a data structure shown below. In the “scenario”, the pieces of data (the protocol A, the protocol B, the protocol C, and the protocol D) stored in the “theater” are treated as “actor”, which are different from the actual data.
Keyword: Pilot, Data: Actor-A (Protocol A)
Keyword: Pilot-B, Data: Actor-B (Protocol B)
Keyword: ECGPrep, Data: Actor-C (Protocol C)
Keyword: FBI, Data: Actor-D (Protocol D)

Further, the “producer” instructs the “director” to start the “scenario”.

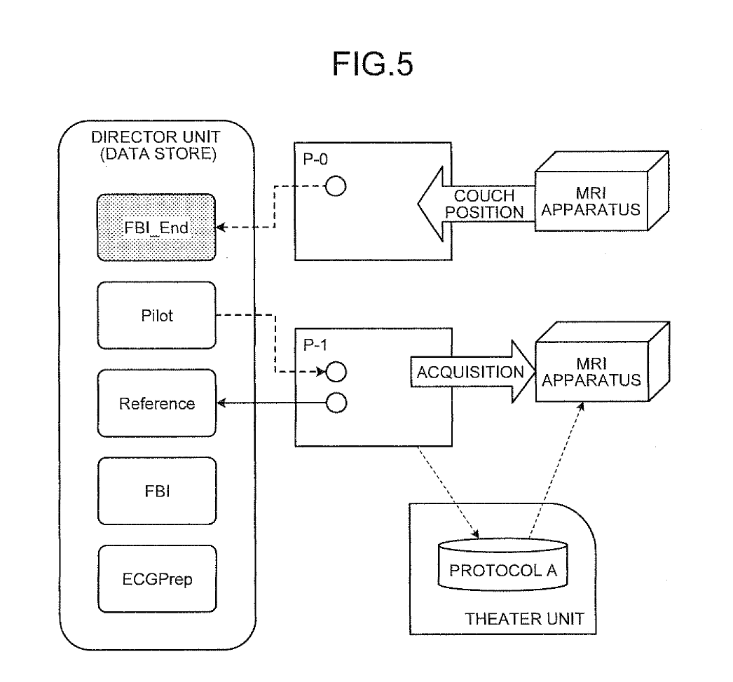

The explanation will continue with reference to FIG. 5. The “director” reads the “scenario” and starts the process (a “scene” or a “performance”) written at the head of the “scenario”. In the first embodiment, the process at the head is “P-0: performance”.

Let us assume that the following information is written in the “scenario”.

P-0: performance (GetCouchPos)

Process: Reads a couch position from the MRI apparatus 100 and records the read couch position

Output: Keyword: FBI_End, Data: couch position

In this situation, for example, the “director” obtains a ‘couch position’ from the couch controlling unit 5 via the interface unit 21 and stores the obtained ‘couch position’ into the “data store” with the keyword ‘FBI_End’.

After that, the “director” executes “P-1: performance” according to the order written in the “scenario”.

Let us assume that the following information is written in the “scenario”.

P-1: performance (Acquire)

Input: Keyword: Pilot

Process: Perform an acquisition process on the actor (i.e., the protocol A) indicated by “Pilot”

Output: Keyword: Reference, Data: the actor corresponding to the input (i.e., the protocol A)

In that situation, the “director” reads the actual data “protocol A” corresponding to the data ‘Actor-A’ registered with the keyword ‘Pilot’ from the “theater” and instructs the image-taking controller 26e to perform an image taking procedure according to the ‘Actor-A’ i.e., the “protocol A”. Accordingly, the image-taking controller 26e controls the image taking procedure so as to acquire a pilot image and to store the acquired pilot image into the “protocol A”. Further, the “director” registers the ‘Actor-A’ corresponding to the data “protocol A” in which the pilot image is stored, into the “data store” with the keyword ‘Reference’.

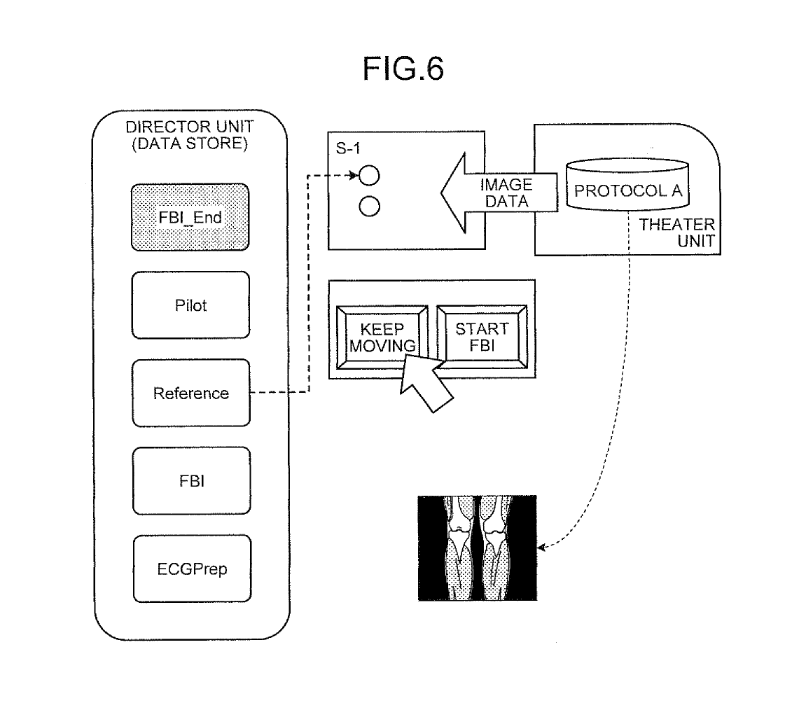

The explanation will continue with reference to FIG. 6. The “director” executes “S-1: scene” according to the order written in the “scenario”.

Let us assume that the following information is written in the “scenario”.

S-1: scene (AutoFBI Pilot)

Input: Keyword: Reference

Process: Display the input image

GUI:

a “keep moving” button

a “start FBI” button

Action:

If the “keep moving” button is selected, start Performance: P-2

If the “start FBI” button is selected, start Scene: S-2.

In that situation, the “director” reads the actual data “protocol A (including the image data of the pilot image)” corresponding to the data ‘Actor-A’ registered with the keyword ‘Reference’ from the “theater” and displays the read pilot image on the display unit 25. Further, the “director” displays the “keep moving” button and the “start FBI” button on the display unit 25, as a GUI for receiving an input operation from the operator. Also, if the “keep moving” button is pressed by the operator via the input unit 24, the “director” executes “P-2: performance”. In contrast, if the “start FBI” button is pressed by the operator via the input unit 24, the “director” executes “S-2: scene”.

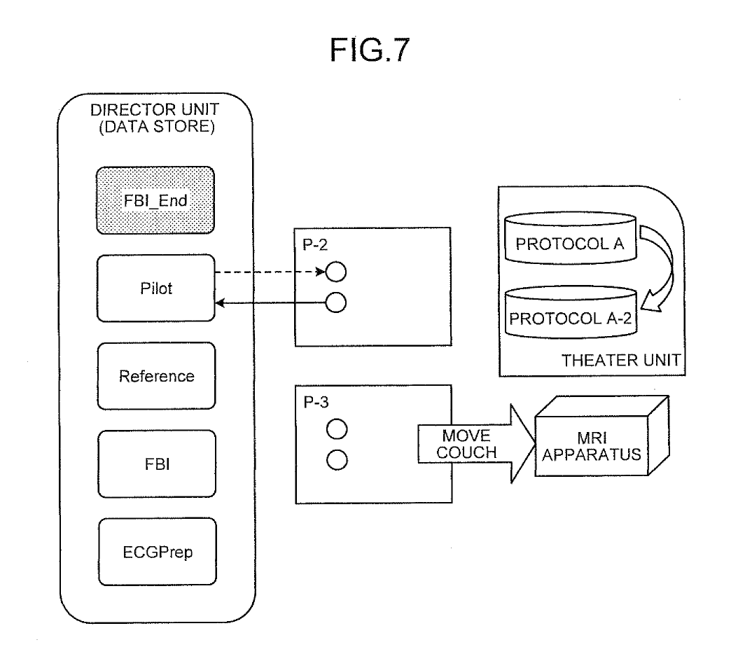

The explanation will continue with reference to FIG. 7. Let us assume that the “keep moving” button is pressed by the operator in “S-1: scene” described above. In that situation, the “director” executes “P-2: performance” according to the order written in the “scenario”.

Let us assume that the following information is written in the “scenario”.

P-2: performance (Duplicate)

Input: Keyword: Pilot

Process: Create a duplicate of the input actor

Output: Keyword: Pilot, Data: the duplicated actor (Protocol A-2)

In that situation, the “director” reads the actual data “protocol A” corresponding to the data ‘Actor-A’ registered with the keyword ‘Pilot’ from the “theater”, creates a duplicate thereof, and stores the created duplicate (a protocol A-2) into the “theater”. Further, the “director” registers the ‘Actor-A’ corresponding to the duplicated data (i.e., the protocol A-2) into the “data store” with the keyword ‘Pilot’.

Subsequently, the “director” executes “P-3: performance” according to the order written in the “scenario”.

Let us assume that the following information is written in the “scenario”.

P-3: performance (MoveCouch)

Operation: Move the couch

Action: Start Performance: P-1

In that situation, for example, the “director” moves the couch 4 by controlling the couch controlling unit 5 via the interface unit 21 and executes “P-1: performance”.

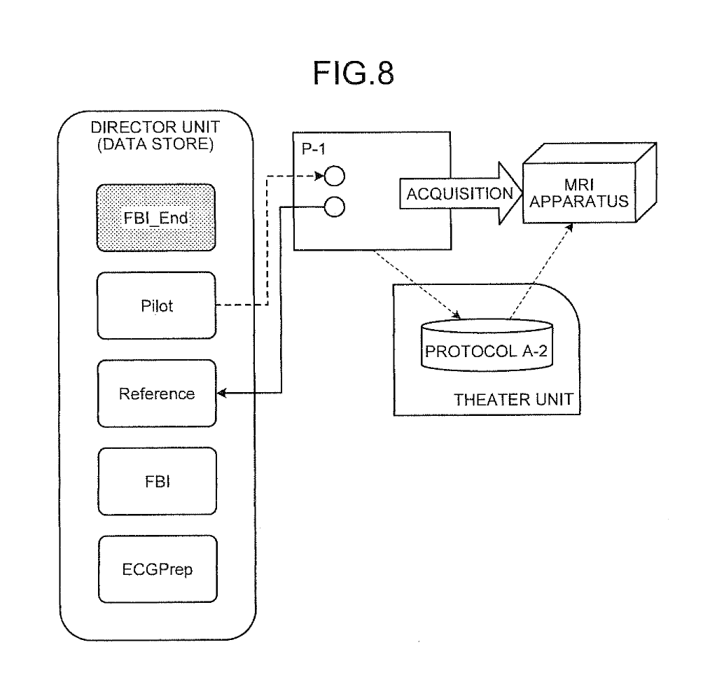

The explanation will continue with reference to FIG. 8. The “director” executes “P-1: performance” according to the order written in the “scenario”.

As described above, the following information is written in the “scenario”.

P-1: performance (Acquire)

Input: Keyword: Pilot

Process: Perform an acquisition process on the actor (i.e., the protocol A) indicated by “Pilot”

Output: Keyword: Reference, Data: the actor corresponding to the input (i.e., the protocol A)

In that situation, the “director” reads the actual data “protocol A-2” corresponding to the data ‘Actor-A’ registered with the keyword ‘Pilot’ from the “theater” and instructs the image-taking controller 26e to perform an image taking procedure according to the ‘Actor-A’ i.e., the “protocol. A-2”. Accordingly, the image-taking controller 26e controls the image taking procedure so as to acquire a pilot image and to store the acquired pilot image into the “protocol A-2”. Further, the “director” registers the ‘Actor-A’ corresponding to the data “protocol A-2” in which the pilot image is stored, into the “data store” with the keyword ‘Reference’.

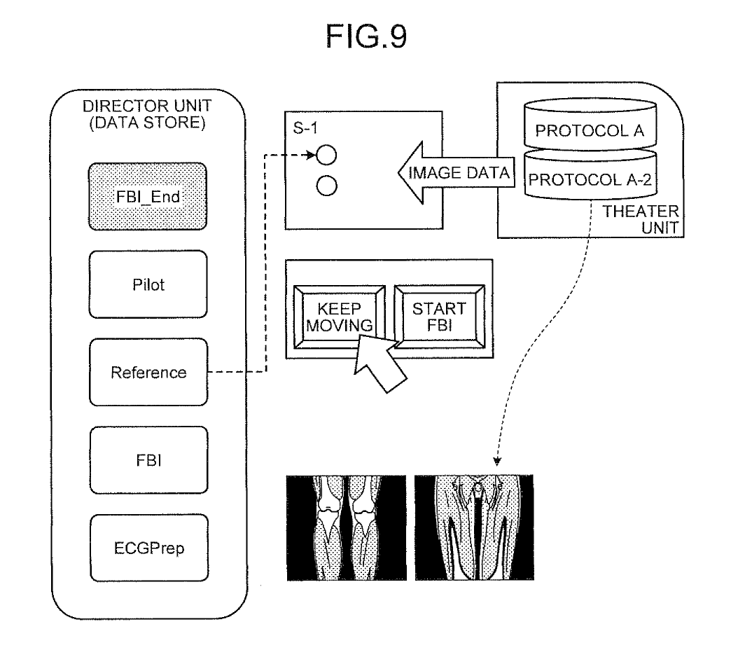

The explanation will continue with reference to FIG. 9. The “director” executes “S-1: scene” according to the order written in the “scenario”.

As described above, the following information is written in the “scenario”.

S-1: scene (AutoFBI_Pilot)

Input: Keyword: Reference

Process: Display the input image

GUI:

a “keep moving” button

a “start FBI” button

Action:

If the “keep moving” button is selected, start Performance: P-2

If the “start FBI” button is selected, start Scene: S-2.

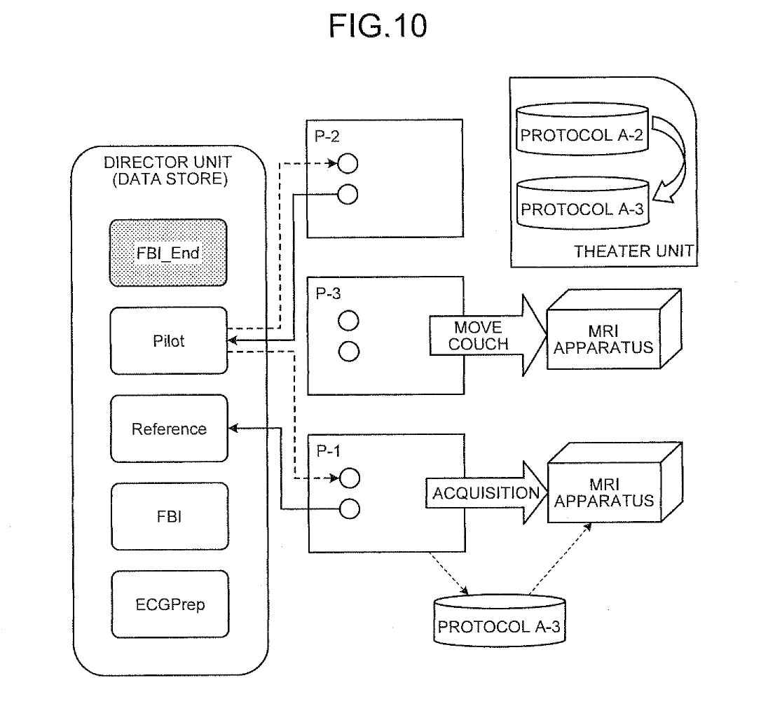

When the ‘keep moving’ button is pressed again by the operator via the input unit 24, the “director” executes “P-2: performance”. After that, as shown in FIG. 10, the “director” repeats the execution of “P-3: performance” and the execution of “P-1: performance”.

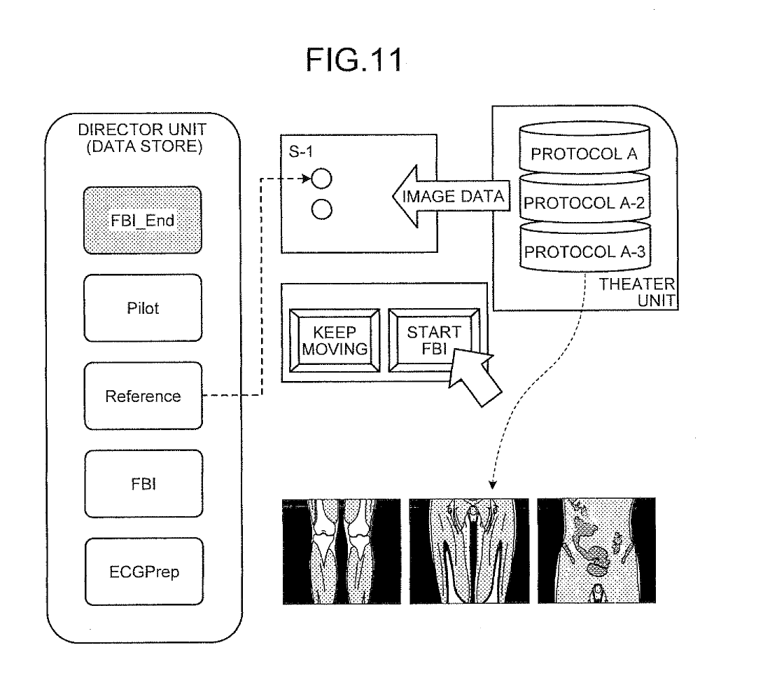

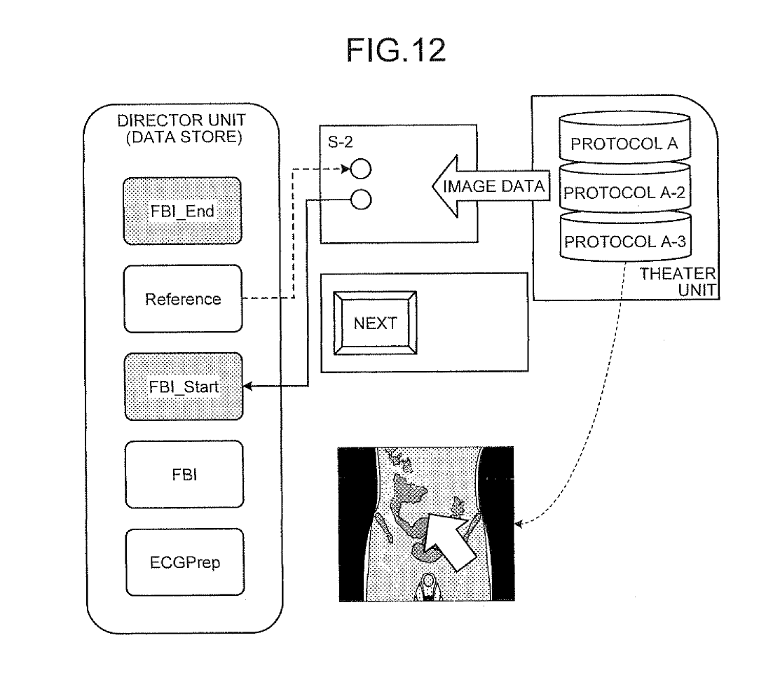

The explanation will continue with reference to FIGS. 11 and 12. Let us assume that the “start FBI” button is pressed by the operator in “S-1: scene” described above. In that situation, the “director” executes “S-2: scene” according to the order written in the “scenario”.

Let us assume that the following information is written in the “scenario”.

S-2: scene (AutoFBI_StartPos)

Input: Keyword: Reference

Process: Display the last image among the input images

Operation: The operator specifies an FBI starting point in the displayed image

GUI:

a “Next” button

Action:

If the “Next” button is selected, start Performance: P-4

Output:

Keyword: FBI_Start, Data: couch position

In that situation, the “director” reads the actual data “protocol A-3” corresponding to the data ‘Actor-A’ registered with the keyword ‘Reference’ from the “theater” and displays the read pilot image on the display unit 25. Further, the “director” displays the “Next” button on the display unit 25, as a GUI for receiving an input operation from the operator.

Further, the “director” receives an operation to specify an FBI starting point performed by the operator in the pilot image displayed on the display unit 25, and also, executes “P-4: performance” if the “Next” button is pressed. In addition, for example, the “director” obtains a ‘couch position’ from the couch controlling unit 5 via the interface unit 21 and stores the obtained ‘couch position’ into the “data store” with the keyword ‘FBI_Start’.

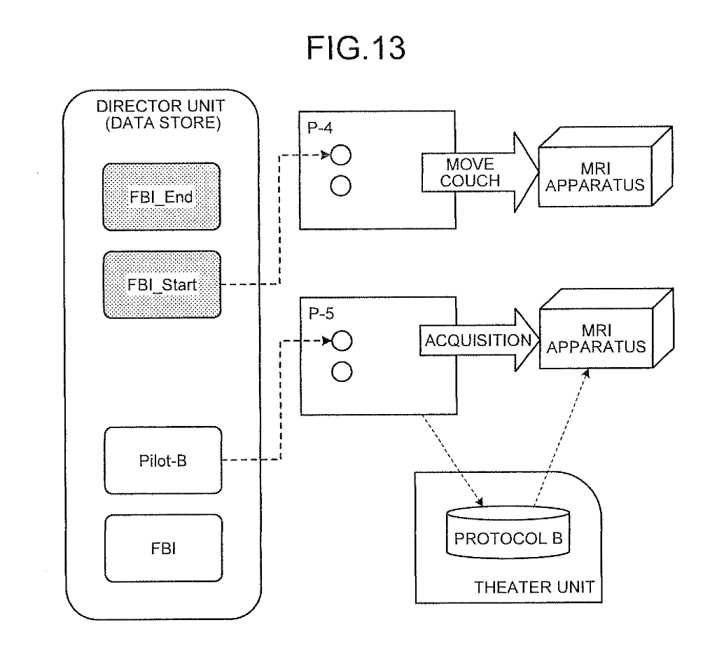

The explanation will continue with reference to FIG. 13. The “director” executes “P-4: performance” according to the order written in the “scenario”.

Let us assume that the following information is written in the “scenario”.

P-4: performance (MoveCouch)

Input: Keyword: FBI_Start

Operation: Move the couch to the input position

In that situation, the “director” reads the couch position registered with the keyword ‘FBI_Start’ from the “data store” and moves the couch 4 to the couch position by, for example, controlling the couch controlling unit 5 via the interface unit 21.

After that, the “director” executes “P-5: performance” according to the order written in the “scenario”.

Let us assume that the following information is written in the “scenario”.

P-5: performance (Acquire)

Input: Keyword: Pilot-B

Process: Perform an acquisition process on the actor (i.e., the protocol B) indicated by “Pilot-B”

In that situation, the “director” reads the actual data “protocol B” corresponding to the data ‘Actor-B’ registered with the keyword ‘Pilot-B’ from the “theater” and instructs the image-taking controller 26e to perform an image taking procedure according to the ‘Actor-B’ i.e., the “protocol B”. Accordingly, the image-taking controller 26e controls the image taking procedure so as to acquire a plan image and to store the acquired plan image into the “Protocol B”.

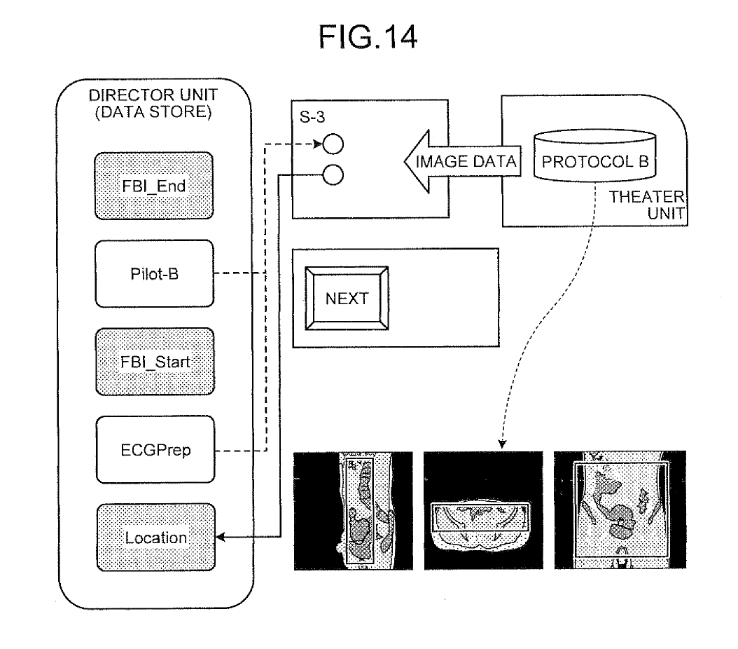

The explanation will continue with reference to FIG. 14. The “director” executes “S-3: scene” according to the order written in the “scenario”.

Let us assume that the following information is written in the “scenario”.

S-3: scene (AutoFBI_Plan)

Input:

Keyword: Pilot-B

Keyword: ECGPrep

Process: Display the input image

Operation: The operator specifies an image taking position in the displayed image

GUI:

a “Next” button

Action:

If the “Next” button is selected start Performance: P-6

Output:

Keyword: Location, Data: image taking position

In that situation, the “director” reads the actual data “protocol B” corresponding to the data ‘Actor-B’ registered with the keyword ‘Pilot-B’ from the “theater” and displays the read plan image on the display unit 25. Further, the “director” displays the ‘Next’ button on the display unit 25, as a GUI for receiving an input operation from the operator.

Further, the “director” receives an operation to specify an image taking position performed by the operator in the plan image displayed on the display unit 25, and also, executes “P-6: performance” if the “Next” button is pressed. On the plan screen shown in FIG. 14, frames for receiving an operation to specify the image taking position are displayed. These frames are defined by the actual data “protocol C” corresponding to the data ‘Actor-C’ registered with the keyword ‘ECGPrep’. In other words, the “director” reads the “protocol C” from the “theater” and displays the information about the frames that has been read on the plan screen. Further, the “director” stores the image taking position received as the input operation into the “data store” with a keyword ‘Location’.

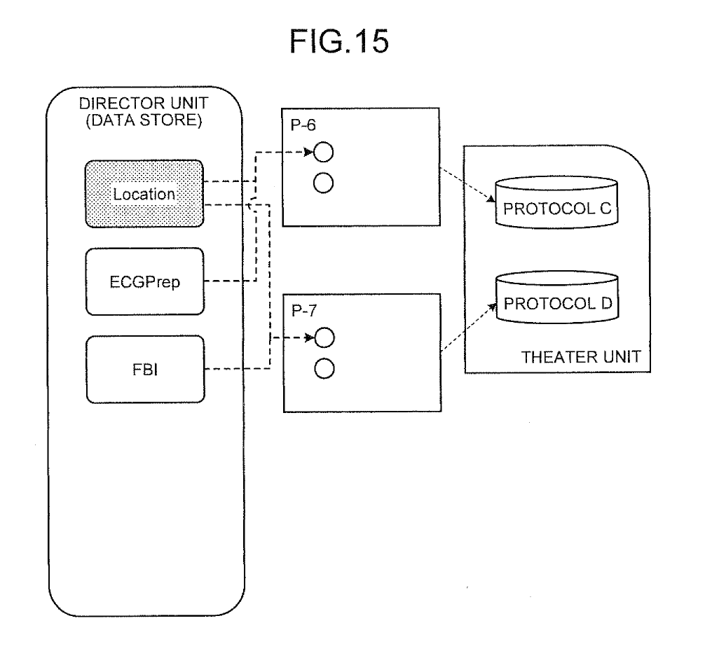

The explanation will continue with reference to FIG. 15. The “director” executes “P-6: performance” according to the order written in the “scenario”.

Let us assume that the following information is written in the “scenario”.

P-6: performance (CopyLocation)

Input:

Keyword: Location

Keyword: ECGPrep

Process: Copy a first input “Location” into the actor (i.e., the protocol C) indicated by a second input “ECGPrep”

In that situation, the “director” reads the image taking position registered with the keyword ‘Location’ from the “data store”, and also, reads the actual data “protocol C” corresponding to the data ‘Actor-C’ registered with the keyword ‘ECGPrep’ from the “theater”. Further, the “director” writes the image taking position having been read into the “protocol C” and stores the protocol C into the “theater”.

After that, the “director” executes “P-7: performance” according to the order written in the “scenario”.

Let us assume that the following information is written in the “scenario”.

P-7: performance (CopyLocation)

Input:

Keyword: Location

Keyword: FBI

Process: Copy a first input “Location” into the actor (i.e., a protocol D) indicated by a second input “FBI”

In that situation, the “director” reads the image taking position registered with the keyword ‘Location’ from the “data store”, and also, reads the actual data “protocol D” corresponding to the data ‘Actor-D’ registered with the keyword ‘FBI’ from the “theater”. Further, the “director” writes the image taking position having been read into the “protocol D” and stores the “protocol D” into the “theater”.

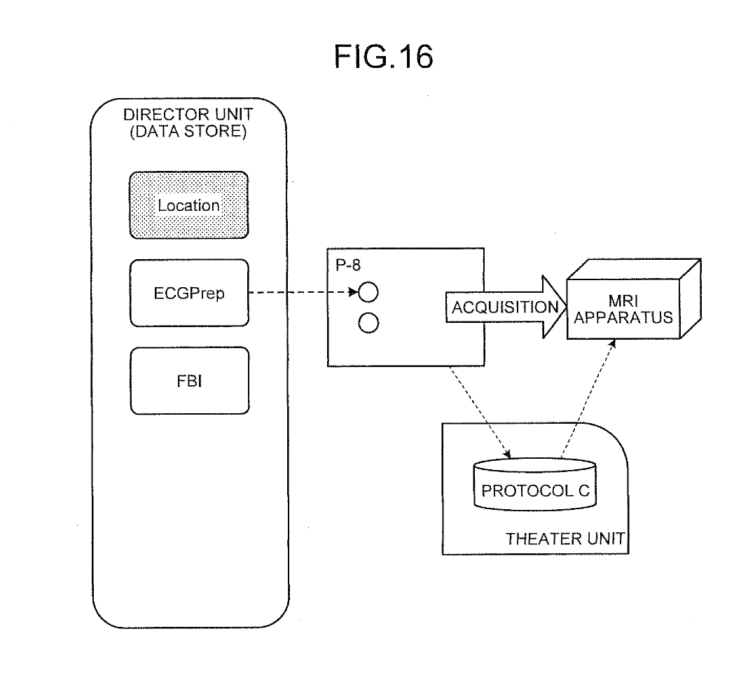

The explanation will continue with reference to FIG. 16. The “director” executes “P-8: performance” according to the order written in the “scenario”.

Let us assume that the following information is written in the “scenario”.

P-8: performance (Acquire)

Input Keyword: ECGPrep

Process: Perform an acquisition process on the actor (i.e., the protocol C) indicated by “ECGPrep”

In that situation, the “director” reads the actual data “protocol C” corresponding to the data ‘Actor-C’ registered with the keyword ‘ECGPrep’ from the “theater” and instructs the image-taking controller 26e to perform an image taking procedure according to the ‘Actor-C’ i.e., the “protocol C”. Accordingly, the image-taking controller 26e controls the image taking procedure so as to acquire an ECG-Prep image and to store the acquired ECG-Prep image into the “protocol C”.

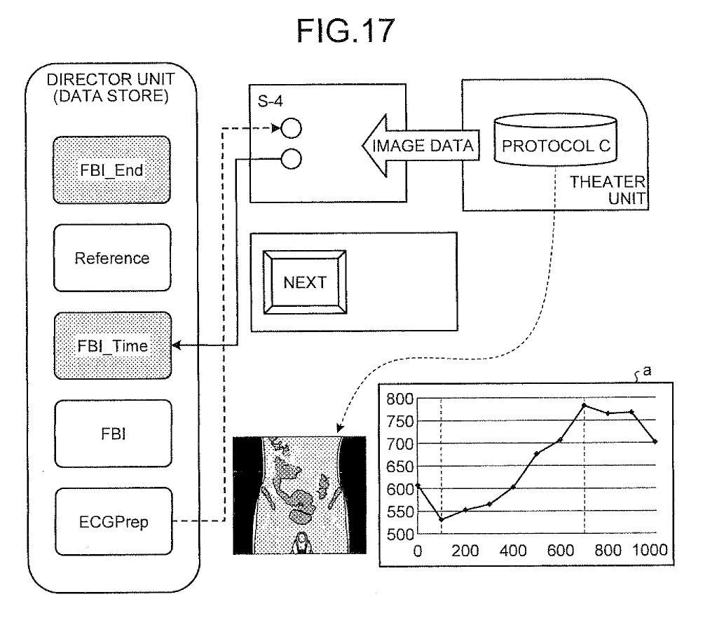

The explanation will continue with reference to FIG. 17. FIG. 17 depicts an example in which optimal temporal phases (i.e., a delay period) are determined by receiving an input operation from the operator. The “director” executes “S-4: scene” according to the order written in the “scenario”.

Let us assume that the following information is written in the “scenario”.

S-4: scene (AutoFBI_ECGPrep)

Input: Keyword: ECGPrep

Process: Extract a feature amount from the input image and display the extracted feature amount in a chart

Operation: The operator selects optimal temporal phases in two places in the chart

GUI:

a “Next” button

Action:

If the “Next button” is selected, start Scene: S-5

Output:

Keyword: FBI_Time, Data: optimal temporal phases (two places)

In that situation, the “director” reads the actual data “protocol C” corresponding to the data ‘Actor-C’ registered with the keyword ‘ECGPrep’ from the “theater” and displays the read ECG-Prep image on the display unit 25. Further, the “director” displays the “Next” button on the display unit 25, as a GUI for receiving an input operation from the operator.

Also, the “director” extracts a feature amount from the read ECG-Prep image and displays a chart “a” on the display unit 25. After that, the “director” receives an operation to select the optimal temporal phases (in two places) performed by the operator in the chart “a” displayed on the display unit 25, and also, executes “S-5: scene” if the “Next” button is pressed. Further, the “director” stores the optimal temporal phases (in the two places) selected by the operator into the “data store” with a keyword ‘FBI_Time’.

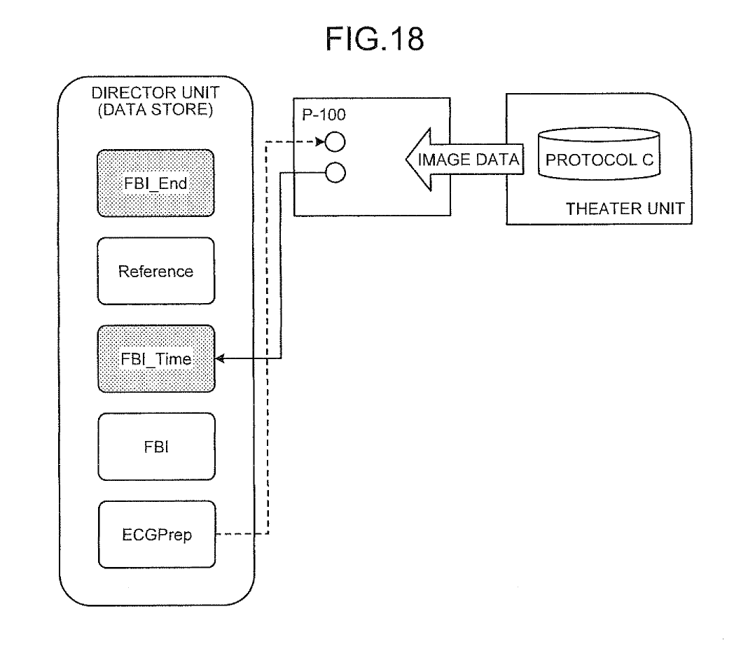

The explanation will continue with reference to FIG. 18. FIG. 18 depicts an example in which the optimal temporal phases (i.e., the delay period) are automatically determined. The “director” executes “P-100: performance” according to the order written in the “scenario”.

Let us assume that the following information is written in the “scenario”.

P-100: performance (CalculateFBITiming)

Input: Keyword: ECGPrep

Process: Extract a feature amount from the input image and automatically calculate the optimal temporal phases

Output: Keyword: FBI_Time, Data: optimal temporal phases (two places)

In that situation, the “director” reads the actual data “protocol C” corresponding to the data ‘Actor-C’ registered with the keyword ‘ECGPrep’ from the “theater”, extracts a feature amount from the read ECG-Prep image, and automatically calculates the optimal temporal phases (in two places). Further, the “director” stores the automatically-calculated optimal temporal phases (in the two places) into the “data store” with the keyword ‘FBI_Time’.

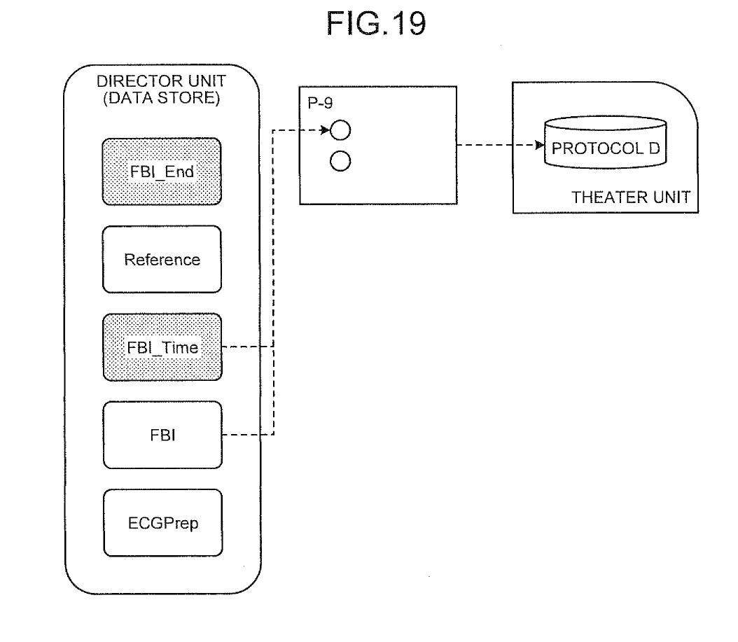

The explanation will continue with reference to FIG. 19. The “director” executes “P-9: scene” according to the order written in the “scenario”.

Let us assume that the following information is written in the “scenario”.

P-9: performance (ApplyFBITiming)

Input:

Keyword: FBI_Time

Keyword: FBI

Process: Set the temporal phases (in the two places) indicated by a first input “FBI_Time” into the actor (i.e., the protocol D) indicated by a second input “FBI”, as a synchronization delay period

In that situation, the “director” reads the optimal temporal phases (in the two places) registered with the keyword ‘FBI_Time’ from the “data store”, and also, reads the actual data “protocol D” corresponding to the data ‘Actor-D’ registered with the keyword ‘FBI’ from the “theater”. Further, the “director” sets the read optimal temporal phases (in the two places) into the “protocol D” and stores the “protocol D” into the “theater”.

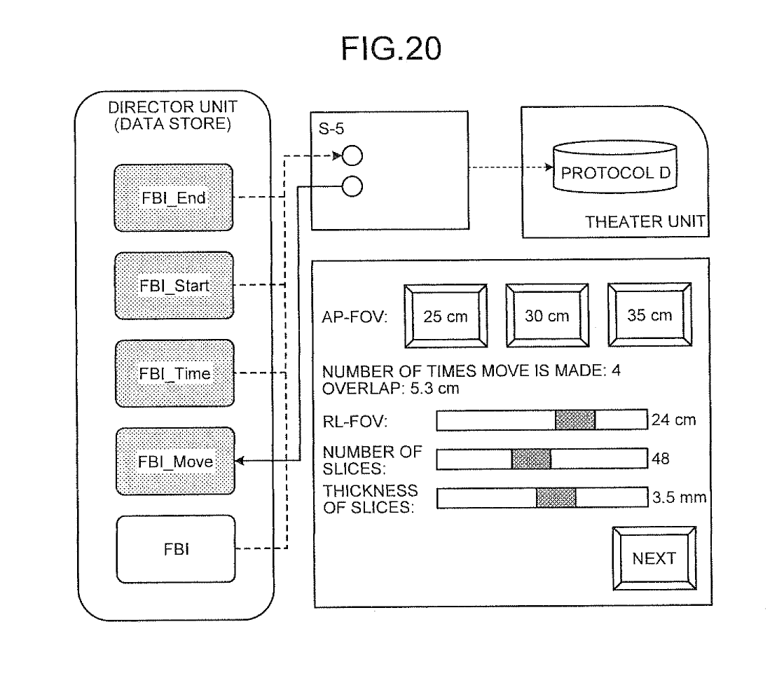

The explanation will continue with reference to FIG. 20. The “director” executes “S-5: scene” according to the order written in the “scenario”.

Let us assume that the following information is written in the “scenario”.

S-5: scene (AutoFBI_Main)

Input:

Keyword: FBI_Start

Keyword: FBI_End

Keyword: FBI_Time

Keyword: FBI

Operation:

The operator selects an FOV in a body-axis direction using the GUI

Process: Based on the selected FOV in the body-axis direction, calculate and display the number of times a move is made and an overlap

Store edited results of an FOV in the horizontal direction, the number of slices, and the thickness of the slices into the actor (i.e., the protocol D) indicated by the “FBI”.

Incorporate the temporal phases indicated by the keyword “FBI_Time” into the image taking condition as the synchronization delay period.

GUI:

A body-axis-direction FOV button (The FOV to be displayed is obtained from the scenario)

Display the number of times a move is made and the overlap

The horizontal-direction FOV (an image taking condition for the actor indicated by the keyword “FBI”)

The number of slices (an image taking condition for the actor indicated by the keyword “FBI”)

The thickness of the slices (an image taking condition for the actor indicated by the keyword “FBI”)

A “Next” button

Action:

If the “Next” button is selected, start Performance: P-10

Output:

Keyword: FBI_Move, Data: couch movement amount, the number of times a move is made

In that situation, the “director” reads the couch position registered with the keyword ‘FBI_End’ from the “data store”. Further, the “director” reads the couch position registered with the keyword ‘FBI_Start’ from the “data store”. Also, the “director” reads the optimal temporal phases (in the two places) registered with the keyword ‘FBI_Time’ from the “data store”. Furthermore, the “director” reads the actual data “protocol D” corresponding to the data ‘Actor-D’ registered with the keyword ‘FBI’ from the “theater”.

Further, the “director” displays an image taking condition editing screen on the display unit 25, as a GUI for receiving an input operation from the operator. On the image taking condition editing screen, information selected according to the purpose of the image taking procedure is displayed, as the information that is necessary for receiving the input operation. For example, as shown in FIG. 19, the following pieces of information are displayed on the image taking condition editing screen: information for receiving a selection of a Field Of View (FOV) in the body-axis direction (AP-FOV); information for receiving a setting for an FOV in the horizontal direction (RL-FOV); information for receiving a setting for the number of slices; information for receiving a setting for the thickness of the slices; and the ‘Next’ button.

Further, when having received an input operation indicating the FOV in the body-axis direction from the operator, the “director” calculates the couch movement amount, the number of times a move is made, and the overlap and displays the number of times a move is made and the overlap on the display unit 25. Further, when the ‘Next’ button is pressed on the image taking condition editing screen displayed on the display unit 25, the “director” stores the information received as the input operation into the actual data “protocol D” corresponding to the ‘Actor-D’.

Further, if the ‘Next’ button is pressed on the image taking condition editing screen displayed on the display unit 25, the “director” stores the couch movement amount and the number of times a move is made that have been calculated, into the “data store” with the keyword ‘FBI_Move’. Further, if the ‘Next’ button is pressed on the image taking condition editing screen displayed on the display unit 25, the “director” executes “P-10: performance”.

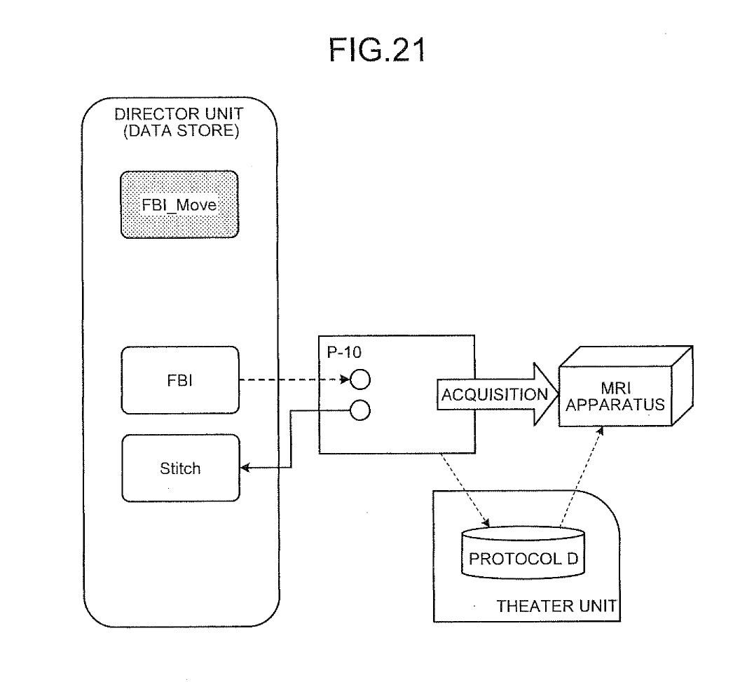

The explanation will continue with reference to FIG. 21. The “director” executes “P-10: performance” according to the order written in the “scenario”.

Let us assume that the following information is written in the “scenario”.

P-10: performance (Acquire)

Input: Keyword: FBI

Process: Perform an acquisition process on the actor (i.e., the protocol D) indicated by “FBI”

Output: Keyword: Stitch, Data: the actor corresponding to the input (i.e., the protocol D)

In that situation, the “director” reads the actual data “protocol D” corresponding to the data ‘Actor-D’ registered with the keyword ‘FBI’ from the “theater” and instructs the image-taking controller 26e to perform an image taking procedure according to the ‘Actor-D’ i.e., the “protocol D”. Accordingly, the image-taking controller 26e controls the image taking procedure so as to acquire an FBI image and to store the acquired FBI image into the “protocol D”. Further, the “director” registers the ‘Actor-D’ corresponding to the data “protocol D” in which the FBI image is stored, into the “data store” with a keyword ‘Stitch’.

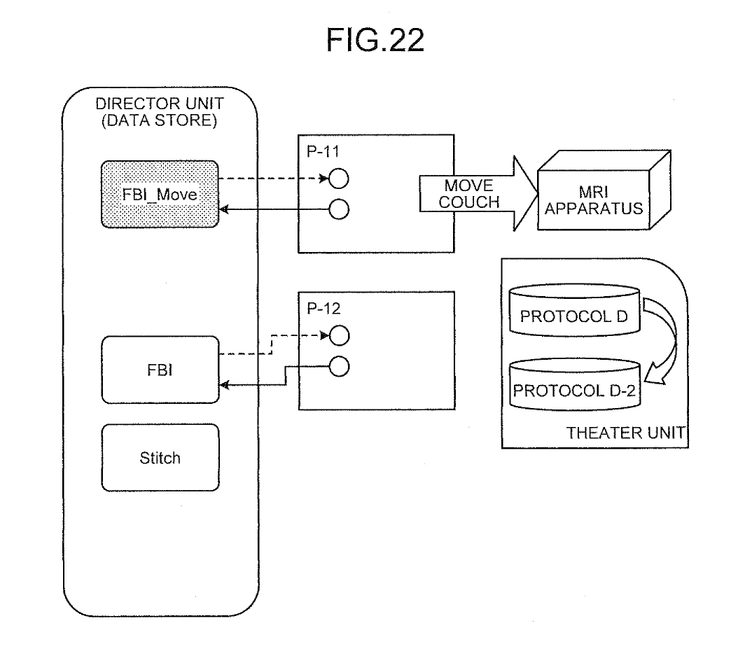

The explanation will continue with reference to FIG. 22. The “director” executes “P-11: performance” according to the order written in the “scenario”.

Let us assume that the following information is written in the “scenario”.

P-11: performance (MoveCouch)

Input: Keyword: FBI_Move

Operation: If the number of times a move is made is smaller than a predetermined value, move the couch. When the number of times has reached the predetermined value, start Performance: P-13.

Output: Keyword: FBI_Move, Data in which the number of times a move is made has been updated

In that situation, the “director” reads the couch movement amount and the value indicating the number of times a move is made that are registered with the keyword ‘FBI_Move’ from the “data store” and, if the number of times a move is made is smaller than the predetermined value, the “director” moves the couch 4 by, for example, controlling the couch controlling unit 5 via the interface unit 21, so that the couch 4 is moved according to the read couch movement amount. Further, the “director” updates the value indicating the number of times a move is made with respect to the data registered with the keyword ‘FBI_Move’ and stores the updated data into the “data store”.

After that, the “director” executes “P-12: performance” according to the order written in the “scenario”.

Let us assume that the following information is written in the “scenario”.

P-12: performance (Duplicate)

Input: Keyword: FBI

Process: Create a duplicate of the input actor

Output: Keyword: FBI, Data: the duplicated actor (i.e., a Protocol D-2)

In that situation, the “director” reads the actual data “protocol D” corresponding to the data ‘Actor-D’ registered with the keyword ‘FBI’ from the “theater”, creates a duplicate thereof, and stores the created duplicate (i.e., the protocol D-2) into the “theater”. Further, the “director” registers the ‘Actor-D’ corresponding to the duplicated data (i.e., the protocol D-2) into the “data store” with the keyword ‘FBI’.

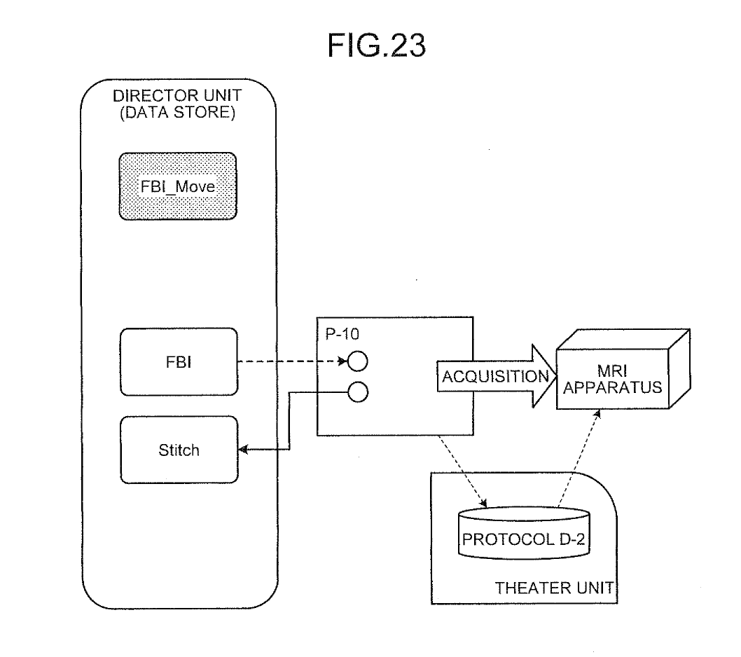

The explanation will continue with reference to FIG. 23. The “director” executes “P-10: performance” according to the order written in the “scenario”.

As described above, the following information is written in the “scenario”.

P-10: performance (Acquire)

Input: Keyword: FBI

Process: Perform an acquisition process on the actor (i.e., the protocol D) indicated by “FBI”

Output: Keyword: Stitch, Data: the actor corresponding to the input (i.e., the protocol D)

In that situation, the “director” reads the actual data “protocol D-2” corresponding to the data ‘Actor-D’ registered with the keyword ‘FBI’ from the “theater” and instructs the image-taking controller 26e to perform an image taking procedure according to the ‘Actor-D’ i.e., the “protocol D-2”. Accordingly, the image-taking controller 26e controls the image taking procedure so as to acquire an FBI image and to store the acquired FBI image into the data “protocol D-2”. Further, the “director” registers the ‘Actor-D’ corresponding to the data “protocol D-2” in which the FBI image is stored, into the “data store” with the keyword ‘Stitch’.

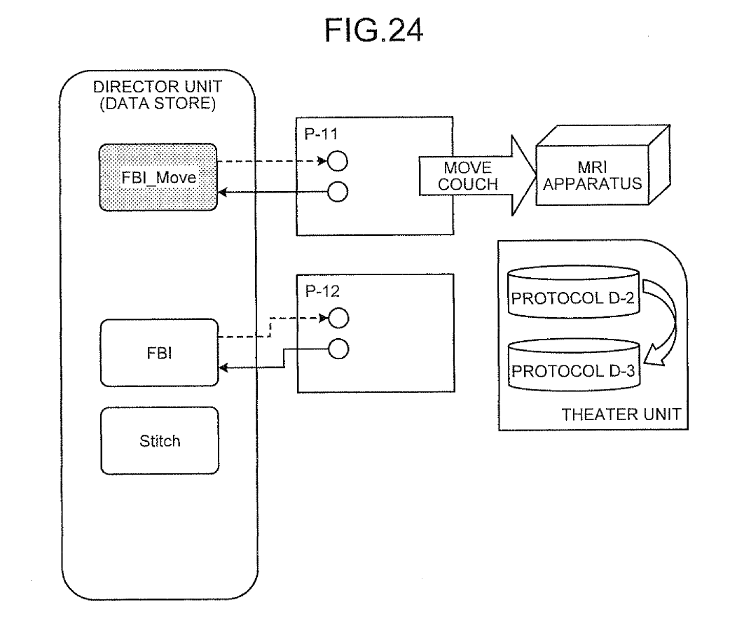

The explanation will continue with reference to FIG. 24. The “director” executes “P-11: performance” according to the order written in the “scenario”.

As described above, the following information is written in the “scenario”.

P-11: performance (MoveCouch)

Input: Keyword: FBI_Move

Operation: If the number of times a move is made is smaller than a predetermined value, move the couch. When the number of times has reached the predetermined value, start Performance: P-13.

Output: Keyword: FBI_Move, Data in which the number of times a move is made has been updated

In that situation, the “director” reads the couch movement amount and the value indicating the number of times a move is made that are registered with the keyword ‘FBI_Move’ from the “data store” and, if the number of times a move is made is smaller than the predetermined value, the “director” moves the couch 4 by, for example, controlling the couch controlling unit 5 via the interface unit 21, so that the couch 4 is moved according to the read couch movement amount. Further, the “director” updates the value indicating the number of times a move is made with respect to the data registered with the keyword ‘FBI_Move’ and stores the updated data into the “data store”.

After that, the “director” executes “P-12: performance” according to the order written in the “scenario”.

As described above, the following information is written in the “scenario”.

P-12: performance (Duplicate)

Input: Keyword: FBI

Process: Create a duplicate of the input actor

Output: Keyword: FBI, Data: the duplicated actor (i.e., a Protocol D-3)

In that situation, the “director” reads the actual data “protocol D-2” corresponding to the data ‘Actor-D’ registered with the keyword ‘FBI’ from the “theater”, creates a duplicate thereof, and stores the created duplicate (i.e., the protocol D-3) into the “theater”. Further, the “director” registers the ‘Actor-D’ corresponding to the duplicated data (i.e., the protocol D-3) into the “data store” with the keyword ‘FBI’.

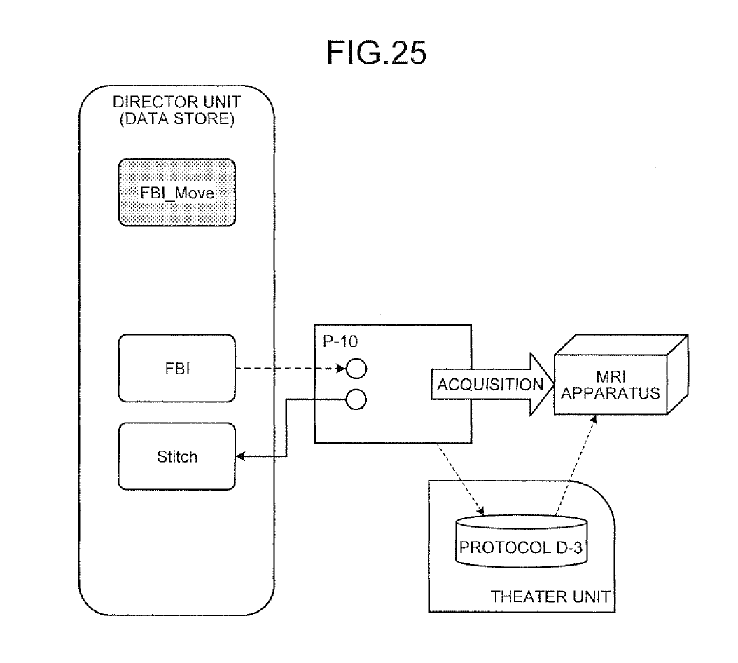

The explanation will continue with reference to FIG. 25. The “director” executes “P-10: performance” according to the order written in the “scenario”.

As described above, the following information is written in the “scenario”.

P-10: performance (Acquire)

Input: Keyword: FBI

Process: Perform an acquisition process on the actor (i.e., the protocol D) indicated by “FBI”

Output: Keyword: Stitch, Data: the actor corresponding to the input (i.e., the protocol D)

In that situation, the “director” reads the actual data “protocol D-3” corresponding to the data ‘Actor-D’ registered with the keyword ‘FBI’ from the “theater” and instructs the image-taking controller 26e to perform an image taking procedure according to the ‘Actor-D’ i.e., the “protocol D-3”. Accordingly, the image-taking controller 26e controls the image taking procedure so as to acquire an FBI image and to store the acquired FBI image into the data “protocol D-3”. Further, the “director” registers the ‘Actor-D’ corresponding to the data “protocol D-3” in which the FBI image is stored, into the “data store” with the keyword ‘Stitch’.

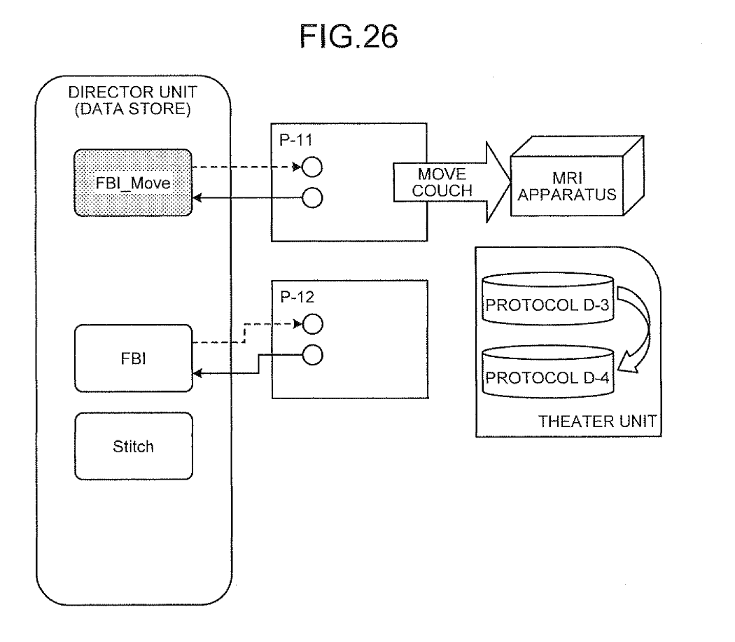

The explanation will continue with reference to FIG. 26. The “director” executes “P-11: performance” according to the order written in the “scenario”.

As described above, the following information is written in the “scenario”.

P-11: performance (MoveCouch)

Input: Keyword: FBI_Move

Operation: If the number of times a move is made is smaller than a predetermined value, move the couch. When the number of times has reached the predetermined value, start Performance: P-13.

Output: Keyword: FBI_Move, Data in which the number of times a move is made has been updated

In that situation, the “director” reads the couch movement amount and the value indicating the number of times a move is made that are registered with the keyword ‘FBI_Move’ from the “data store” and, if the number of times a move is made is smaller than the predetermined value, the “director” moves the couch 4 by, for example, controlling the couch controlling unit 5 via the interface unit 21, so that the couch 4 is moved according to the read couch movement amount. Further, the “director” updates the value indicating the number of times a move is made with respect to the data registered with the keyword ‘FBI_Move’ and stores the updated data into the “data store”.

After that, the “director” executes “P-12: performance” according to the order written in the “scenario”.

As described above, the following information is written in the “scenario”.

P-12: performance (Duplicate)

Input: Keyword: FBI

Process: Create a duplicate of the input actor

Output: Keyword: FBI, Data: the duplicated actor (i.e., a Protocol D-4)

In that situation, the “director” reads the actual data “protocol. D-3” corresponding to the data ‘Actor-D’ registered with the keyword ‘FBI’ from the “theater”, creates a duplicate thereof, and stores the created duplicate (i.e., the protocol D-4) into the “theater”. Further, the “director” registers the ‘Actor-D’ corresponding to the duplicated data (i.e., the protocol D-4) into the “data store” with the keyword ‘FBI’.

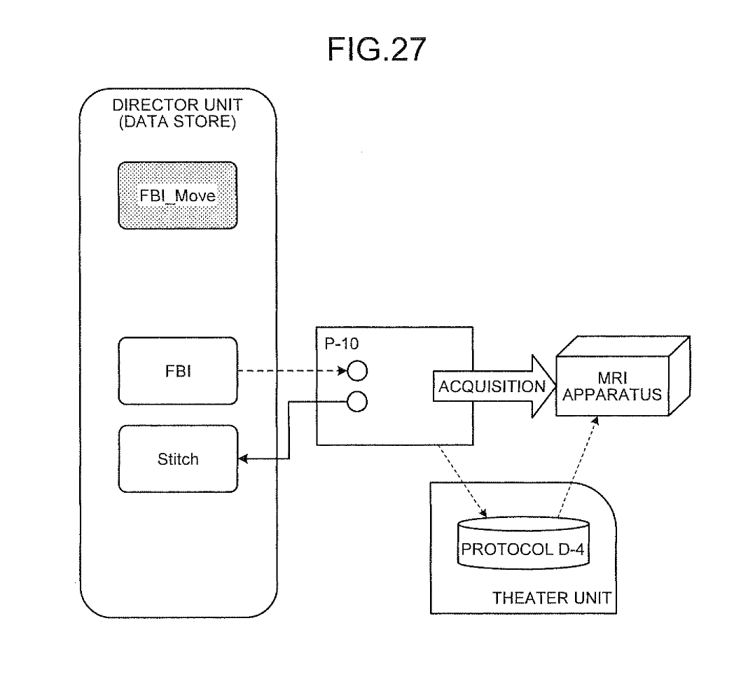

The explanation will continue with reference to FIG. 27. The “director” executes “P-10: performance” according to the order written in the “scenario”.

As described above, the following information is written in the “scenario”.

P-10: performance (Acquire)

Input: Keyword: FBI

Process: Perform an acquisition process on the actor (i.e., the protocol D) indicated by “FBI”

Output: Keyword: Stitch, Data: the actor corresponding to the input (i.e., the protocol D)

In that situation, the “director” reads the actual data “protocol D-4” corresponding to the data ‘Actor-D’ registered with the keyword ‘FBI’ from the “theater” and instructs the image-taking controller 26e to perform an image taking procedure according to the ‘Actor-D’ i.e., the “protocol D-4”. Accordingly, the image-taking controller 26e controls the image taking procedure so as to acquire an FBI image and to store the acquired FBI image into the data “protocol D-4”. Further, the “director” registers the ‘Actor-D’ corresponding to the data “protocol D-4” in which the FBI image is stored, into the “data stere” with the keyword ‘Stitch’.

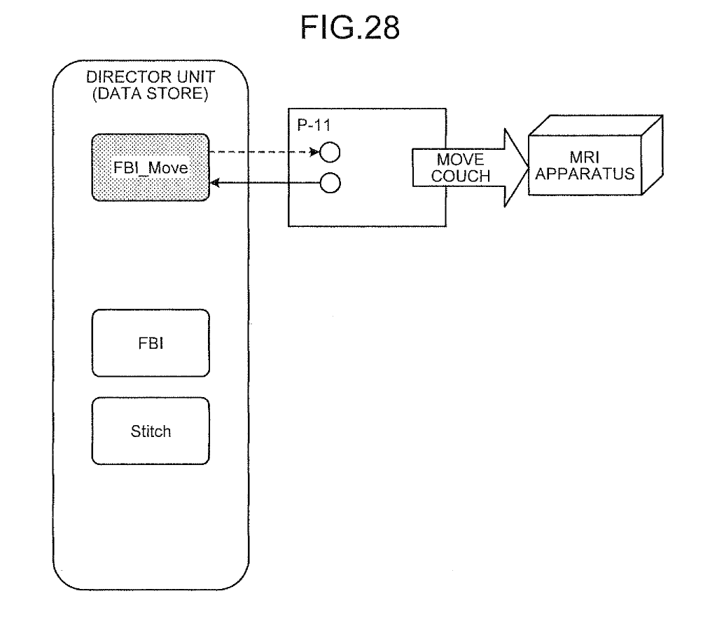

The explanation will continue with reference to FIG. 28. The “director” executes “P-11: performance” according to the order written in the “scenario”.

As described above, the following information is written in the “scenario”.

P-11: performance (MoveCouch)

Input: Keyword: FBI_Move

Operation: If the number of times a move is made is smaller than a predetermined value, move the couch. When the number of times has reached the predetermined value, start Performance: P-13.

Output: Keyword: FBI_Move, Data in which the number of times a move is made has been updated

In that situation, the “director” reads the couch movement amount and the value indicating the number of times a move is made that are registered with the keyword ‘FBI_Move’ from the “data store” and, if the number of times a move is made is smaller than the predetermined value, the “director” moves the couch 4 by, for example, controlling the couch controlling unit 5 via the interface unit 21, so that the couch 4 is moved according to the read couch movement amount.

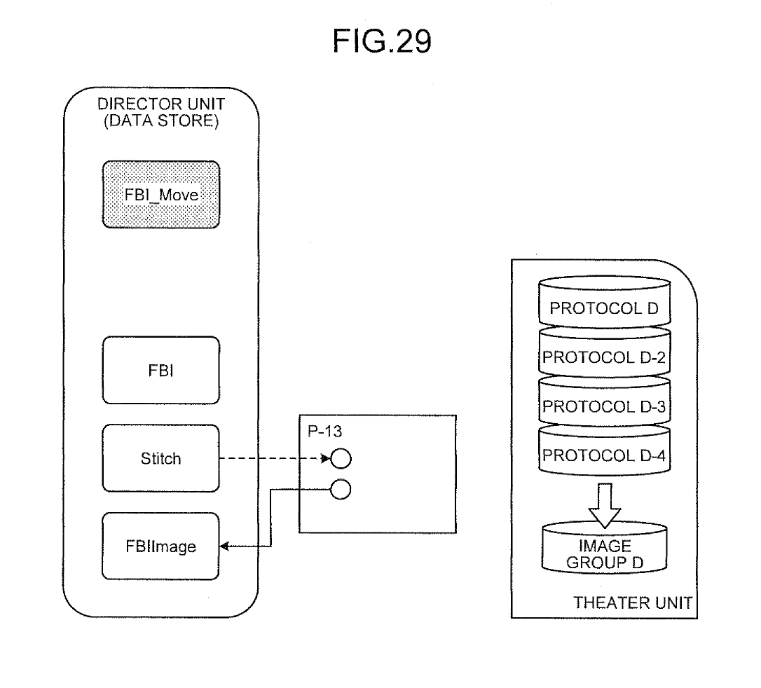

The explanation will continue with reference to FIG. 29. When the number of times has reached the predetermined value, the “director” executes “Performance: P-13” according to the order written in the “scenario”.

Let us assume that the following information is written in the “scenario”.

P-13: performance (StitchFBIImage)

Input: Keyword: Stitch

Process: Perform a maximum value projecting process on the actor (i.e., the protocols D, D-2, D-3, and D-4) indicated by “Stitch” and stitch the images together

Output: “FBIImage”, Data: a group of images stitched together

In that situation, the “director” reads the actual data “protocol D”, “protocol D-2”, “protocol D-3”, and “protocol D-4” corresponding to the data (i.e., the ‘Actor-D’) registered with the keyword ‘Stitch’ from the “theater” and performs a maximum value projecting process. Further, the “director” stores the group of images stitched together D into the “theater” and registers the group of images D into the “data store” with the keyword ‘FBIImage’.

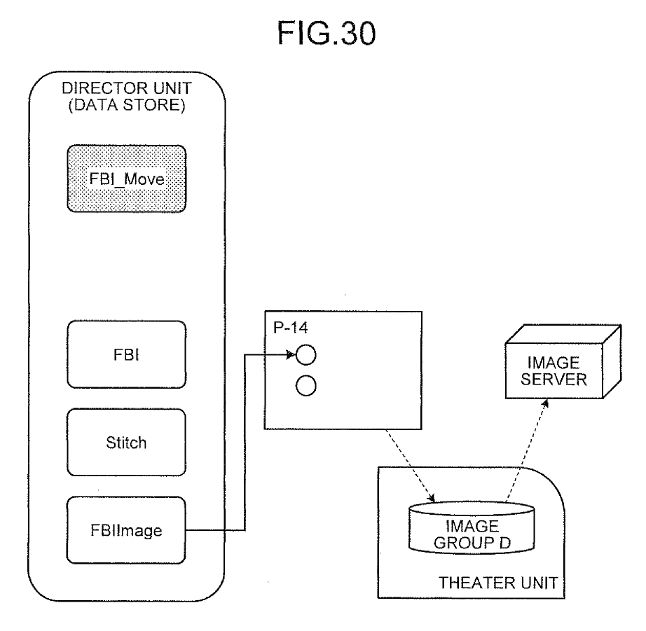

The explanation will continue with reference to FIG. 30. The “director” executes “P-14: performance” according to the order written in the “scenario”.

Let us assume that the following information is written in the “scenario”.

P-14: performance (Transfer)

Input: Keyword: FBIImage

Operation: Transfer the image data of the actor (i.e., the group of images D) indicated by the keyword ‘FBIImage’ to an image server

In that situation, the “director” reads the group of image data registered with the keyword ‘FBIImage’ from the “theater” and transfers the group of image data D having been read to the image server.

As explained above, the MRI apparatus 100 according to the first embodiment has stored, in the scenario storage 23b, the “scenario” for executing the plurality of processes contained in the image taking procedure. The “scenario” is a program in which the processes are classified into the “scenes” for which an input operation from the operator is received and the “performances” for which an input operation is not received, while the processes are associated with one another according to the order in which the processes are to be executed during the image taking procedure. Further, the MRI apparatus 100 includes the scenario controller 26b. When an image-taking start instruction is received, the scenario controller 26b exercises control so that the execution of the “scenario” is started and so that the processes are executed according to the order during the image taking procedure. Further, the “scenario” is arranged so that, when any of the “scenes” is executed, the information selected according to the purpose of the image taking procedure is displayed on the display unit 25, as the operation screen for receiving the input operation.

With these arrangements, according to the first embodiment, it is possible to improve operability during the image taking procedure employing the MRI apparatus 100. In other words, when the scenario controller 26b controls the execution of the “scenario”, the processes contained in the image taking procedure are automatically executed according to the order during the image taking procedure. The “scenario” is arranged so that, during any of the “scenes” for which an input operation from the operator is received, the operation screen displaying the information selected according to the purpose of the image taking procedure is displayed so as to receive the input operation from the operator. With this arrangement, for example, even if the operator does not have advanced knowledge of parameters, the operator is able to select and set an appropriate parameter according to the purpose of the image taking procedure and the stage of the processes. Consequently, the operation is simplified.

Further, according to the first embodiment, the processes contained in the image taking procedure are separated in a detailed manner by being classified into the “scenes” and the “performances”. Further, the keywords are defined for the data exchanged between the processes, so that the scenario controller 26b causes the data to be exchanged between the processes by using the keywords. With these arrangements, for example, even if the contents of a part of the processes are modified, no change will occur in the input/output data relationship with the processes that precede and follow the modified process. In addition, it will be sufficient to modify only a part of the “scenes” and/or a part of the “performances” that corresponds to the modified process. Consequently, it is possible to flexibly address modifications made to the contents of the processes.

Further, the MRI apparatus 100 according to the first embodiment stores therein a plurality of “scenarios” according to the purposes of the image taking procedures. The scenario controller 26b reads, out of the scenario storage 23b, one of the “scenarios” corresponding to the image taking procedure instructed in the image-taking start instruction and starts the execution of the read “scenario”. With this arrangement, the MRI apparatus 100 is able to address various purposes of the image taking procedures. In addition, because each of the “scenarios” is prepared according to the purpose of the image taking procedure, it is possible to simplify the operation screen. In addition, it is also possible to limit selectable functions with the “scenarios”. It is therefore possible to prevent the situation where the selection of parameters made by the operator fluctuates. As a result, it is possible to maintain the quality of the taken images at a certain level.

The “scenarios” may be construed as a type of programming that defines not only the flows of the operations but also the GUIs.

It is possible to embody the disclosed technical features in other various modes besides the ones described in the first embodiment.

### [Editing the Scenarios]

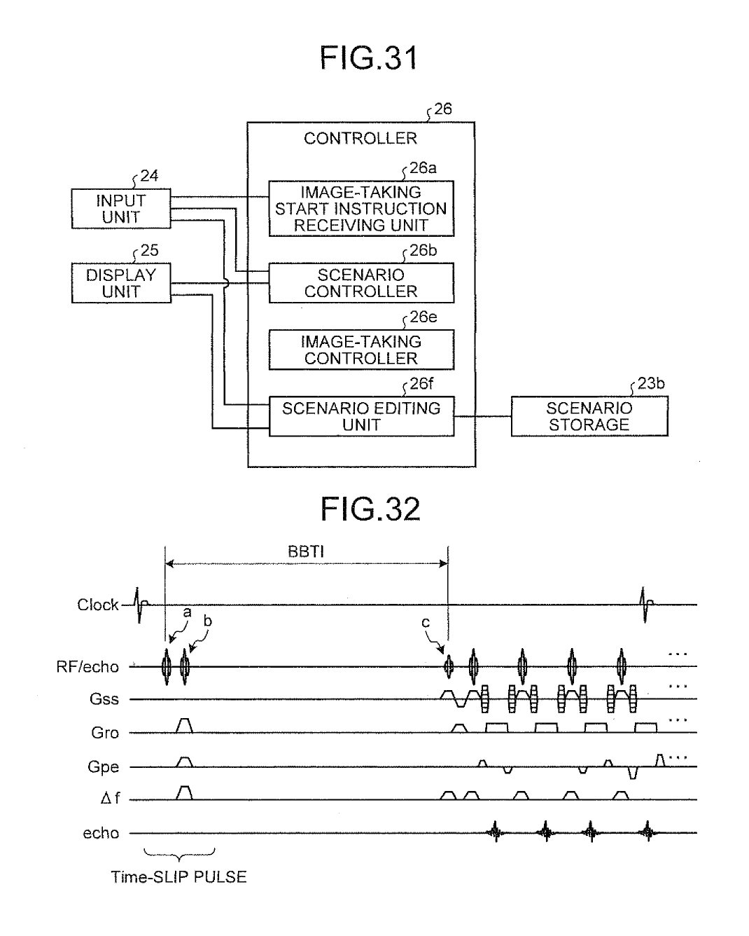

In the first embodiment described above, the “scenario” is stored in advance in the scenario storage 23b, in the form of the file written in, for example, the XML. In this situation, as illustrated in FIG. 31, the MRI apparatus 100 may be configured so that the controller 26 further includes a scenario editing unit 26f. FIG. 31 is a block diagram of the controller 26 according to the second embodiment.

The scenario editing unit 26f receives editing to a “scenario” and stores the “scenario” reflecting the received editing into the scenario storage 23b. For example, the scenario editing unit 26f displays an editing screen for editing the “scenario” on the display unit 25. Further, for example, the scenario editing unit 26f receives an input operation performed on the editing screen by the operator via the input unit 24. Furthermore, for example, the scenario editing unit 26f causes the received input operation to be reflected in the “scenario” and stores the “scenario” reflecting the input operation into the scenario storage 23b. As explained here, the operator is able to modify the contents of the “scenario” and is able to modify the contents without modifying the software. The “scenario” does not necessarily have to be written in the XML. The operator who creates and edits the “scenario” is able to arbitrarily select the language in which the “scenario” is written according to the mode in which the “scenario” is used.

### [The Operation Screen Displaying the Information Selected According to the Purpose of the Image Taking Procedure]

Further, in the first embodiment, the “image taking procedure performed on a leg by using the FBI method” is used as an example of the purpose of the image taking procedure. For instance, the situation was explained where, when the process “S-5: scene” illustrated in FIG. 20 is executed, the information selected according to the purpose of the image taking procedure is displayed on the display unit 25 as the image taking condition editing screen. In other words, according to the purpose of the image taking procedure and the stage of the process “S-5: scene”, the MRI apparatus 100 selects ‘body-axis-direction FOV’, ‘the number of times a move is made’, ‘overlap’, ‘horizontal-direction FOV’, ‘the number of slices’, and ‘the thickness of the slices’ and displays only the selected information.

To “display only the selected information” means that the MRI apparatus 100 does not display such items that do not have a reason to be displayed in terms of the purpose of the image taking procedure and the stage of the processes and that may make the image taking editing screen more complicated when displayed. Accordingly, the sentence is not meant to exclude the possibility of displaying information other than the selected information such as general information required during the operation (e.g., the “Next” button).

Further, as the “selected information”, the MRI apparatus 100 may display items (hereinafter, “attention items”) to which an attention should be paid during the operation. For example, the operator who creates and edits the “scenario” selects, in advance, the attention items that are considered desirable to be displayed in terms of the purpose of the image taking procedure and the stage of the processes, so that the “scenario” is written in such a manner that the attention items are displayed on a screen displayed on the display unit 25 during the scene.

Further, the disclosed technical features are not limited to the examples described in the first embodiment. For instance, if a “scenario” is written according to another purpose of image taking procedure, the MRI apparatus 100 displays, on the display unit 25, an operation screen in which different information is selected according to the purpose of the image taking procedure and the stage of the processes. In other words, because each of the “scenarios” is written according to the purpose of the image taking procedure, it is possible to introduce an operation screen to which appropriate restrictions are applied in correspondence with the purpose of the image taking procedure.

For instance, let us assume that one of the purposes of image taking procedures is “to regulate the speeder direction during a mammography image taking procedure”. In that situation, the operator who creates and edits the “scenario” writes an image taking condition editing screen that displays only options that are necessary with regard to the speeder direction, an image taking condition editing screen that prohibits selecting a speeder direction itself, an image taking condition editing screen that does not display the options for a speeder direction, in the “scenario”. Accordingly, when the MRI apparatus 100 executes the processes according to the “scenario”, the image taking condition editing screens described above are displayed on the display unit 25, so that the operator naturally makes a selection to regulate the speeder direction.

Further, as another example, let us assume that one of the purposes of image taking procedures is “to limit the resolution of the image” from a viewpoint of image management. In that situation, the operator who creates and edits the “scenario” writes the “scenario” so that buttons in each of which an FOV and a matrix are combined (e.g., [25 centimeters/256 matrix] and [20 centimeters/192 matrix]) are displayed on an image taking condition editing screen. Accordingly, when the MRI apparatus 100 executes the processes according to the “scenario”, the image taking condition editing screen described above is displayed on the display unit 25, so that the operator naturally selects one of the options [25 centimeters/256 matrix] and [20 centimeters/192 matrix].

These are methods for realizing simple operations by introducing appropriate restrictions, and these methods may be considered as a type of navigation. When restrictions are partially introduced, there is a possibility that the operator may feel dissatisfied; however, when restrictions are introduced on a large scale, the method functions as navigation and is able to realize a simple operation for the operator. Also, from a viewpoint of eliminating fluctuations caused by human factors, the method is effective.

[Advantages of Controlling the Image Taking Procedure with the “Scenario”]

As explained in the first embodiment, in the “scenario”, the protocol information that is dealt with in the processes is defined as the “actor”, which is different from the actual data, so that the processes performed on the “actor” are written in advance. With this arrangement, during the image taking procedure using the “scenario”, it is possible to write, in advance, the processes to be performed on obtained data before the actual data is obtained and to automate the processes contained in the image taking procedure.

For example, generally speaking, it is not possible to select a reference image until an image has been reconstructed, because the image being the selection target does not exist at the earlier stage; however, when the “scenario” is used, the image to be reconstructed is defined as an “actor”, so that a selecting process performed on the “actor” is written in advance in a process at a stage later than the reconstructing process. As a result, it is possible to write, in advance, the process to select the reference image at a stage earlier than when the image is reconstructed, i.e., before the image taking procedure. Consequently, it is possible to automate the processes contained in the image taking procedure.

For example, the “scenario” can be written, in advance, so as to read “the actor B selects the center image in the actor A as a reference image” or “the actor C selects the center image in the actor A and the center image in the actor B as reference images”. In that situation, it is assumed that the number of slices is fixed.

As another example, the “scenario” can be written so as to read “the center image in the actor A is displayed in the first frame, whereas the center image in the actor B is displayed in the second frame, and an orthogonal cross section is set for the center image in the actor B”. In that situation, it is possible to set, in advance, an identical cross section or an orthogonal cross section with respect to the reference image.

Further, let us imagine that, for example, element technology for automating determination of a cross section is established. In that situation, it is possible to write a “scenario” in such a manner that a cross section is automatically determined in a “performance” so that the operator is prompted to confirm the cross section in a “scene”. Alternatively, the “scene” where the operator is prompted to confirm the cross section may be omitted from the “scenario”. As explained here, it is possible to edit the “scenario” as appropriate so as to dynamically fit established element technology.

### [An Image Taking Procedure Employing a Time-SLIP Method]

In another exemplary embodiment, the clinical application scenario can be a medical examination flow for sequentially executing: a pilot scan for determining a position; a prep scan for determining a Black Blood Traveling Time (BBTI); and a non-contrast-enhanced MRA scan for performing an image taking procedure when the determined BBTI has elapsed. In this exemplary embodiment, the scenario controller 26b displays, on an operation screen, information for determining an image taking position including a position determining image obtained by the pilot scan, at a time after the pilot scan and before the prep scan. Also, the scenario controller 26b displays, on an operation screen, information for supporting the determination of the BBTI, at a time after the prep scan and before the non-contrast-enhanced MRA scan.

More specifically, the first embodiment is explained on the assumption that the “scenario” includes the pilot image taking procedure, the plan image taking procedure, the ECG-Prep image taking procedure, and the FBI image taking procedure; however, the exemplary embodiments are not limited to this example. As another example, a situation will be explained in which a “scenario” includes a pilot image taking procedure, a plan image taking procedure, a Black Blood Traveling Time (BBTI)-Prep image taking procedure, and a Time Spatial Labeling Inversion Pulse (Time-SLIP) image taking procedure. FIG. 32 is a drawing for explaining the scenario according to this exemplary embodiment.

During a Time-SLIP image taking procedure, Time-SLIP pulses (indicated by characters “a” and “b” in FIG. 32) are applied, so that the blood flowing into an image taking region is labeled. In other words, the Time-SLIP image taking procedure is an image taking procedure during which an Arterial Spin Labeling (ASL) pulse is applied for the purpose of selectively enhancing or suppressing the labeled blood by labeling the blood flowing into the image taking region. By using the Time-SLIP image taking procedure, it is possible to selectively enhance or suppress the signal strength of the blood reaching the image taking region when an inversion time (TI) has elapsed. In this situation, a Time-SLIP pulse is applied when a predetermined delay period has elapsed after a clock or an R wave in an ECG signal.

As shown in FIG. 32, the Time-SLIP pulses include a non-region-selecting inversion pulse “a” and a region-selecting inversion pulse “b”. For the non-region-selecting inversion pulse “a”, it is possible to select whether the pulse should be applied or not. The Time-SLIP pulses include at least the region-selecting inversion pulse “b”.

When the blood flowing into the image taking region is labeled by applying the region-selecting inversion pulse “b”, the signal strength becomes higher (or lower, if the non-region-selecting inversion pulse “a” is not applied) in the region where the blood reaches after the BBTI has elapsed.

In other words, as shown in FIG. 32, the BBTI is a time period between the region-selecting inversion pulse “b” and a first Radio Frequency (RF) pulse “c”. Because the time difference between the non-region-selecting inversion pulse “a” and the region-selecting inversion pulse “b” is small enough to be negligible, the time period between the non-region-selecting inversion pulse “a” and the first RF pulse “c” is indicated as the BBTI in FIG. 32.

For this reason, the MRI apparatus 100 determines an optimal BBTI by, for example, performing a preparatory image taking procedure. In other words, the BBTI-Prep image taking procedure is a preparatory image taking procedure that is performed while varying the BBTI, for the purpose of determining the optimal BBTI.

For example, by using 60 milliseconds, 120 milliseconds, and 180 milliseconds as mutually-different BBTIs, the MRI apparatus 100 performs the image taking procedure a plurality of times, while using a different one of the BBTIs each time. Further, the MRI apparatus 100 displays a plurality of two-dimensional images obtained during the image taking procedures on the display unit 25, prompts the operator to select one of the two-dimensional images, and determines that the BBTI used for obtaining the two-dimensional image selected by the operator is the BBTI to be used during the Time-SLIP image taking procedure. In that situation, among from the plurality of two-dimensional images displayed on the display unit 25, the operator selects the two-dimensional image in which, for example, the blood vessels are best rendered. Alternatively, the MRI apparatus 100 may perform an image analysis or the like on the plurality of two-dimensional images so as to determine that the BBTI obtained as a result of the image analysis or the like is the BBTI to be used in the Time-SLIP image taking procedure. After that, the MRI apparatus 100 acquires, for example, a three-dimensional image by performing the TIME-SLIP image taking procedure.

In the first embodiment, the execution of the scenario according to the first embodiment is explained with reference to FIGS. 4 to 30. The MRI apparatus 100 may perform the BBTI-Prep image taking procedure instead of the EGG-Prep image taking procedure according to the first embodiment and perform the Time-SLIP image taking procedure instead of the FBI image taking procedure according to the first embodiment.

For instance, the first embodiment is explained with reference to FIG. 17 where the “director” displays the chart by executing the “scene” so that the selection is received from the operator. Similarly, the “director” may execute a “scene” so as to display, on the display unit 25, the two-dimensional images acquired during the BBTI-Prep image taking procedure executed in a “performance” and so as to receive a selection from the operator. Further, the “director” may store the BBTI used for obtaining the two-dimensional image selected by the operator, into the “data store” with a keyword ‘BBTI’, for example.

### [A Post-Processing Procedure]

The scenario storage 23b included in the MRI apparatus 100 according to another exemplary embodiment stores therein a program for executing a plurality of processes contained in a post-processing procedure performed on data acquired during an image taking procedure, while the processes are classified into first processes for which an input operation from the operator is received and second processes for which an input operation is not received, and the processes are associated with one another according to an order in which the processes are to be executed during the post-processing procedure. Further, when a start instruction to start the post-processing procedure is received, the scenario controller 26b exercises control so that the execution of the program is started and the processes are executed according to the order. When executing any of the first processes, the program displays, on a display unit, information selected according to the purpose of the post-processing procedure, as an operation screen for receiving the input operation.

More specifically, in the exemplary embodiments described above, the “scenario” is a program for executing the plurality of types of processes contained in the image taking procedure; however, the exemplary embodiments are not limited to this example. For example, the “scenario” may be a program for executing a plurality of processes contained in a post-processing procedure performed on data acquired during an image taking procedure. In this situation, examples of the post-processing procedure include a post-processing procedure to generate a volume rendering image and a post-processing procedure to generate a Maximum Intensity Projection (MIP) image, from the data acquired during an image taking procedure.

As an example, the post-processing procedure to generate an MIP image will be explained. The post-processing procedure to generate an MIP image includes the number of times of projections, the projection direction, and a region to be cut out from volume data, as conditions for performing the post-processing procedure. In this situation, the number of times of projections and the projection direction are conditions (“pre-set conditions”) that are determined without receiving an input operation from the operator. In contrast, the cut-out region is a condition determined by receiving an input operation from the operator. For example, the operator specifies a specific blood vessel as the cut-out region.

As explained here, the post-processing procedure to generate the MIP image contains a condition specifying process to receive the specification of the cut-out region and a generating process to generate the MIP image by using the pre-set condition and the condition specified by the operator.

Accordingly, for example, the MRI apparatus 100 stores therein a flow in which the condition specifying process and the generating process are arranged in an order, as one scenario. Further, when this scenario is specified by the operator and a start instruction is received, the MRI apparatus 100 displays, during the condition specifying process, an operation screen for receiving a specification of the cut-out region, as a “scene”. Further, the MRI apparatus 100 performs the MIP image generating process as a “performance”, by using the cut-out region specified during the condition specifying process and the other conditions (i.e., the number of times of projections, the projection direction, etc.) that are pre-set.

### [An Image Taking Procedure in a Precise Examination Mode]

As another example, the “scenario” may contain an “image taking procedure in a precise examination mode” for which it is determined whether the procedure should be executed according to a result of an image taking procedure performed at a preceding stage.

In that situation, the “scenario” contains the image taking procedure at the preceding stage and the image taking procedure in the precise examination mode. For example, the MRI apparatus 100 stores therein a flow in which the preceding-stage image taking procedure and the precise-examination-mode image taking procedure are arranged in an order, as one scenario. Further, when this scenario is specified by the operator and a start instruction is received, the MRI apparatus 100 displays, between the preceding-stage image taking procedure (e.g., a T1 image taking procedure and a T2 image taking procedure) and the precise-examination-mode image taking procedure, two-dimensional images acquired during the T1 image taking procedure and the T2 image taking procedure as a “scene”, and also, displays information for prompting the operator to select whether the precise-examination-mode image taking procedure should be executed, on an operation screen. For example, the MRI apparatus 100 displays the two-dimensional images together with pressing buttons to select “execute the precise examination mode” or “not execute the precise examination mode” on the operation screen. Further, according to the selection made by the operator, the MRI apparatus 100 determines whether the precise examination mode should be executed.

For instance, if the precise examination mode is to be executed, the MRI apparatus 100 further displays an operation screen for receiving a specification of a Region Of Interest (ROI) as a “scene” and executes the precise-examination-mode image taking procedure in a “performance” that follows, according to the specification by the operator.

[An Implementation with a Console Apparatus and/or Cloud Computing]

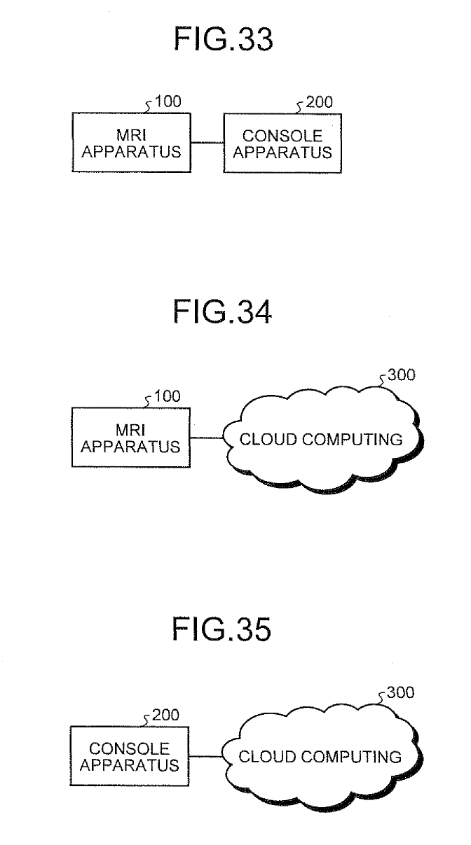

The exemplary embodiments above are explained on the assumption that the computer system 20 in the MRI apparatus 100 includes the functional units within the controller 26 and the storage 23 so as to execute the “scenario”; however, the exemplary embodiments are not limited to this example. FIGS. 33 to 35 are drawings for explaining exemplary configurations according to other embodiments.

For example, as shown in FIG. 33, the computer system 20 in the MRI apparatus 100 and a console apparatus 200 may execute a “scenario” in collaboration with each other.

For example, when the “scenario” is a program for executing a plurality of processes contained in a post-processing procedure, the console apparatus 200 includes the scenario controller 26b, the protocol information storage 23a, the scenario storage 23b, and the input/output information storage 23c. Further, the console apparatus 200 receives data acquired by the MRI apparatus 100 from the MRI apparatus 100 and executes the plurality of processes contained in the post-processing procedure. Alternatively, another arrangement is acceptable in which the console apparatus 200 executes a part of the program executed according to the “scenario” so that the load is distributed. As yet another example, the console apparatus 200 alone may execute the “scenario”.

As another example, as shown in FIG. 34, the MRI apparatus 100 may execute the “scenario” by utilizing hardware, software, and data that are stored in a separate location as in, for example, cloud computing 300.

As yet another example, as shown in FIG. 35, the console apparatus 200 may execute the “scenario” by utilizing hardware, software, and data that are stored in the cloud computing 300.

### [Other Medical Image Diagnosis Apparatuses]

In the exemplary embodiments described above, the MRI apparatus is mainly explained as the medical image diagnosis apparatus that executes the “scenario”; however, the exemplary embodiments are not limited to this example. For instance, another example with an X-ray CT apparatus can be explained as follows. The X-ray CT apparatus performs a preliminary image taking procedure over a large region by performing, for example, helical scanning, displays an image acquired during the image taking procedure as a “scene”, and displays an operation screen for receiving a specification of, for example, a Field Of View (FOV). Further, the X-ray CT apparatus performs a main image taking procedure that follows, according to the specified FOV.

By using the medical image diagnosis apparatus according to at least one of the exemplary embodiments described above, it is possible to improve operability during the image taking procedures.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus comprising:
a storage configured to store therein a program for executing a plurality of processes contained in an image taking procedure or a plurality of processes contained in a post-processing procedure performed on data acquired during the image taking procedure, while the processes are classified into a first group for which an input operation from an operator is received and a second group for which the input operation is not received, and the processes are associated with one another according to an order in which the processes are to be executed during the image taking procedure or the post-processing procedure; and
an execution controller configured to exercise control so that, when a start instruction to start the image taking procedure or the post-processing procedure is received, the execution of the program is started and the processes are executed according to the order, wherein
when executing a process classified into the first group, the program displays, on a display unit, information selected according to a purpose of the image taking procedure or the post-processing procedure, as an operation screen for receiving the input operation.

2. The medical image diagnosis apparatus according to claim 1, wherein
the storage stores therein a plurality of programs according to purposes of the image taking procedure or the post-processing procedure, and
the execution controller reads one of the programs corresponding to the image taking procedure or the post-processing procedure instructed in the start instruction out of the storage and starts the execution of the read program.

3. The medical image diagnosis apparatus according to claim 1, wherein
in the program, a keyword indicating data exchanged between the processes is defined, and
the execution controller causes the data to be exchanged between the processes by using the keyword.

4. The medical image diagnosis apparatus according to claim 1, further comprising: an editing unit configured to receive editing to the program and store the program reflecting the received editing into the storage.

5. The medical image diagnosis apparatus according to claim 1, wherein
the program executes the plurality of processes contained in the image taking procedure that employs a Fresh Blood Imaging (FBI) method, and
the plurality of processes include an electrocardiogram (ECG)-Prep image taking procedure for determining a delay period for an FBI image taking procedure.

6. The medical image diagnosis apparatus according to claim 1, wherein
the program executes the plurality of processes contained in the image taking procedure that employs a Time Spatial Labeling Inversion Pulse (Time-SLIP) method, and
the plurality of processes include a Black Blood Traveling Time (BBTI)-Prep image taking procedure for determining a BBTI for a Time-SLIP image taking procedure.

7. A medical image diagnosis apparatus comprising:
an image taking unit configured to sequentially execute a plurality of types of image taking procedures on a subject;
a storage configured to store therein a plurality of medical examination flows as clinical application scenarios (CASs) in which the plurality of types of image taking procedures are arranged in an order, to store therein a plurality of image taking conditions necessary for executing the image taking procedures, while classifying the image taking conditions into image taking conditions for which an input operation from an operator is received and image taking conditions for which the input operation is not received, and to store therein timing with which an operation screen for receiving the input operation is displayed during the medical examination flows;
a receiving unit configured to receive, from the operator, a start instruction to start a specified one of the plurality of clinical application scenarios; and
a controller configured to, when having received the start instruction, display an operation screen for receiving the input operation at the stored timing while a corresponding one of the medical examination flows is being executed and configured to ensure that an image taking parameter of the image taking conditions set by the input operation and the image taking conditions for which the input operation is not received are reflected in an image taking procedure performed after an input is made on the operation screen.

8. The medical image diagnosis apparatus according to claim 7, wherein
the clinical application scenario specified through the receiving unit is a medical examination flow for sequentially executing: a pilot scan for determining a position; a prep scan for determining a delay period from an R wave; and a non-contrast-enhanced Magnetic Resonance Angiography (MRA) scan for performing an image taking procedure when the determined delay period has elapsed,
the controller displays, on an operation screen, information for determining an image taking position including a position determining image obtained by the pilot scan, at a time after the pilot scan and before the prep scan, and
the controller displays, on an operation screen, information for supporting the determination of the delay period from the R wave, at a time after the prep scan and before the non-contrast-enhanced MRA scan.

9. The medical image diagnosis apparatus according to claim 7, wherein
the clinical application scenario specified through the receiving unit is a medical examination flow for sequentially executing: a pilot scan for determining a position, a prep scan for determining a BBTI, and a non-contrast-enhanced MRA scan for performing an image taking procedure when the determined BBTI has elapsed,
the controller displays, on an operation screen, information for determining an image taking position including a position determining image obtained by the pilot scan, at a time after the pilot scan and before the prep scan, and
the controller displays, on an operation screen, information for supporting the determination of the BBTI, at a time after the prep scan and before the non-contrast-enhanced MRA scan.

10. The medical image diagnosis apparatus according to claim 7, wherein the storage stores the plurality of clinical application scenarios in an external file.

11. A controlling method comprising:
exercising control over a program for executing a plurality of processes contained in an image taking procedure or a plurality of processes contained in a post-processing procedure performed on data acquired during the image taking procedure, while the processes are classified into a first group for which an input operation from an operator is received and a second group for which the input operation is not received, and the processes are associated with one another according to an order in which the processes are to be executed during the image taking procedure or the post-processing procedure, and exercising control so that, when a start instruction to start the image taking procedure or the post-processing procedure is received, the execution of the program is started and the processes are executed according to the order, wherein
when executing a process classified into the first group, the program displays, on a display unit, information selected according to a purpose of the image taking procedure or the post-processing procedure, as an operation screen for receiving the input operation.

12. The controlling method according to claim 11, wherein the program of which the execution is started and that corresponds to the image taking procedure or the post-processing procedure instructed in the start instruction is selected out of a plurality of programs.

13. The controlling method according to claim 11, wherein
in the program, a keyword indicating data exchanged between the processes is defined, and
the data is exchanged between the processes by using the keyword.

14. The controlling method according to claim 11, wherein editing to the program is received.

15. The controlling method according to claim 11, wherein
the program executes the plurality of processes contained in the image taking procedure that employs an FBI method, and
the plurality of processes include an ECG-Prep image taking procedure for determining a delay period for an FBI image taking procedure.

16. The controlling method according to claim 11, wherein
the program executes the plurality of processes contained in the image taking procedure that employs a Time-SLIP) method, and
the plurality of processes include a BBTI-Prep image taking procedure for determining a BBTI for a Time-SLIP image taking procedure.
